(19) United States
(12) Patent Application Publication
(10) Pub. No.: US 2024/0123232 A1
(43) Pub. Date: Apr. 18, 2024

(54) INTEGRATING STIMULATION THERAPY, PATIENT MANAGEMENT, AND EXTERNAL PATIENT MONITORING

(71) Applicant: INSPIRE MEDICAL SYSTEMS, INC., Golden Valley, MN (US)

(72) Inventors: Nicholas Mairs, Minneapolis, MN (US); Brody Belland, St. Louis Park, MN (US); Kent Lee, Minneapolis, MN (US); John Rondoni, Plymouth, MN (US); Aaron Veicht, Vadnais Heights, MN (US); Donovan Fellows, Lake Elmo, MN (US)

(73) Assignee: INSPIRE MEDICAL SYSTEMS, INC., Golden Valley, MN (US)

(21) Appl. No.: 18/278,263

(22) PCT Filed: Feb. 23, 2022
(86) PCT No.: PCT/US2022/017517

§ 371 Date: Aug. 22, 2023

Related U.S. Application Data
(60) Provisional application No. 63/152,672, filed on Feb. 23, 2021.

(51) Int. Cl. A61N 1/36; A61N 1/372

(52) U.S. Cl. CPC A61N 1/3611; A61N 1/36139; A61N 1/3615; A61N 1/37223; A61N 1/37247

(57) ABSTRACT

A method and system for integrating stimulation therapy, patient management, and external patient monitoring.

### BACKGROUND

Some implantable medical devices may communicate with external devices to provide information regarding operation of the implantable medical device within the patient, status of the patient, and the like.

### BRIEF DESCRIPTION OF THE DRAWINGS

FIG. 1 is a block diagram schematically representing an example patient management system including a patient virtual check-in portion.

FIGS. 2-7 are each a diagram schematically representing example patient management clinician user interfaces.

FIG. 8 is a block diagram schematically representing a patient survey.

FIG. 9 is a block diagram schematically representing an example control portion.

FIG. 10 is a block diagram schematically representing an example user interface.

FIG. 11 is a block diagram schematically representing an example patient management system.

FIG. 12 is a block diagram schematically representing an example patient management engine.

FIG. 13 is a diagram schematically representing an example patient management user interface including nightly utilization information tools.

FIG. 14 is a diagram schematically representing an example patient management user interface including therapy effectiveness and outcomes.

FIG. 15A is a block diagram of an example patient device including a patient app.

FIG. 15B is a block diagram of an example patient survey engine.

FIG. 16 is a flow diagram schematically representing an example method of patient management.

FIG. 17A is a block diagram schematically representing an example control portion.

FIG. 17B is a block diagram schematically representing an example user interface.

FIG. 18 is a block diagram schematically representing an example method (and/or example device) for patient care in treating sleep disordered breathing (SDB), which includes aspects of patient management, stimulation therapy, and third party diagnostic/monitoring.

FIG. 19 is a block diagram schematically representing example sensing parameters associated with at least a third party diagnostic/monitoring portion.

FIG. 20 is a block diagram schematically representing example determined parameters associated with at least a third party diagnostic/monitoring portion.

FIG. 21 is a block diagram schematically representing an example resource.

FIG. 22 is a block diagram schematically representing an example method (and/or example device) for integrating a patient management input and a third-party diagnostic/monitoring input into stimulation therapy.

FIG. 23 is a block diagram schematically representing an example method (and/or example device) for integrating a stimulation therapy input and a third-party diagnostic/monitoring input into patient management.

FIG. 24A is a block diagram schematically representing an example stimulation therapy portion.

FIG. 24B is a block diagram schematically representing an example patient management portion.

FIG. 25 is a block diagram schematically representing an example method (and/or example device) for stimulation therapy including closed-loop feedback from a patient management input and a third-party diagnostic/monitoring input.

FIG. 26 is a block diagram schematically representing an example method (and/or example device) for patient management including closed-loop feedback from a stimulation therapy input and a third-party diagnostic/monitoring input.

FIG. 27A is a block diagram schematically representing an example control portion.

FIG. 27B is a block diagram schematically representing an example user interface.

FIG. 28 is a flow diagram schematically representing an example method of patient care for treating sleep disordered breathing (SDB).

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to integrating information from different sources and/or pathways, etc. to enhance patient care in treating sleep disordered breathing (SDB). In some such examples, the different sources and/or pathways may comprise patient management information, stimulation therapy information, and/or third party diagnostic/monitoring information. In some examples, integrating such information may enhance efficacy of stimulation therapy and/or of patient management. The third party diagnostic/monitoring information may be obtained from third party diagnostic/monitoring devices, which may communicate with the devices used to perform patient management and/or devices used to perform stimulation therapy. It will be understood that at least some examples of the present disclosure use the disparate sources of information as a close loop feedback to take actions, such as delivering stimulation therapy, performing patient management actions, adjusting monitoring, and the like. Moreover, each action in turn, may produce further feedback communicated to the various devices and therapy, monitoring, management elements hosted among or on such devices.

At least some examples of the present disclosure are directed to facilitating patient care. In some examples, a method and/or system to facilitate patient care may comprise arrangements which provide for enhanced communication and workflows between and among: (1) a patient and a clinician regarding the status or progress of their therapy and/or evaluation as a patient candidate; and (2) multiple clinicians (e.g. caregiver entities) forming a patient care team. In some examples, a device manufacturer or device service provider may also communicate via such workflows with the care team and/or the patient to facilitate patient care and clinician performance.

Among other aspects, the example methods and/or systems may comprise a virtual check-in, which is engageable on a mobile app (on a user interface of a computing device) and which provides feedback from the patient to the clinician regarding their usage and experience in using a therapy device. Once received, this patient data may at last partially drive clinician workflows on user interfaces adapted to facilitate patient care for a plurality of patients. Among other features, these workflows driven via user interfaces may facilitate a clinician in quickly identifying patients which warrant faster or deeper attention to help the patient achieve desirable treatment outcomes. Similarly, these workflows (via the user interfaces) facilitate communication and coordination among members (e.g. clinicians) of the care team which represent different types of entities (e.g. sleep study center, medical clinic, surgical facility, etc.) treating the patient.

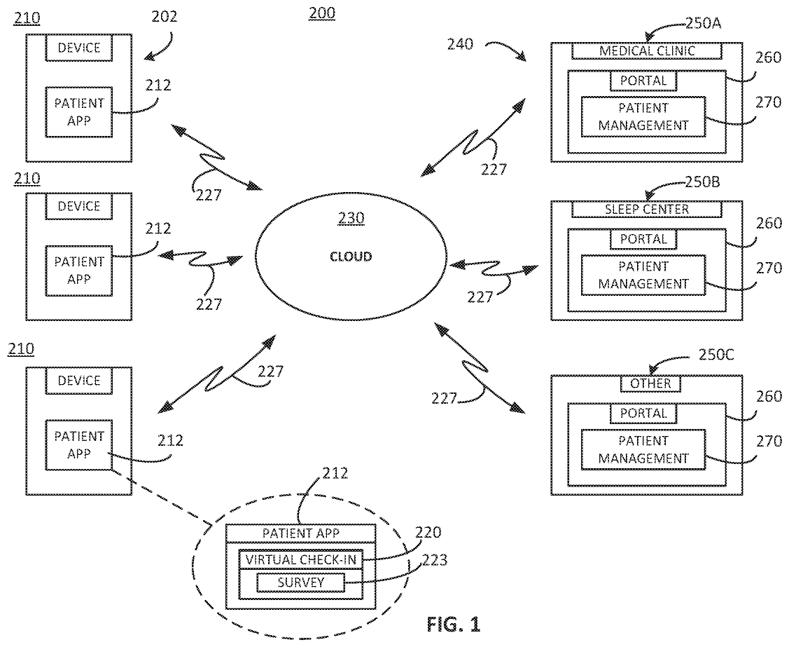

These features and attributes, and additional features and attributes, are described below in association with at least FIGS. 1-10.

At least some examples of the present disclosure are directed to a method and/or apparatus to perform patient care, patient management, and the like. In some examples, a method and/or system to facilitate patient care may comprise arrangements which provide for enhanced communication and patient management between and among: (1) a patient and a clinician regarding the status or progress of their therapy and/or evaluation as a patient candidate; and (2) multiple clinicians (e.g. caregiver entities) forming a patient care team. In some examples, a device manufacturer or device service provider may also communicate with the care team and/or the patient to facilitate patient care and clinician performance. In some examples, the device manufacturer or device service provider also may operate, supply, maintain a processing resource (e.g. cloud server) to provide an arrangement, framework, pathway, etc. by which patient management may be performed with related information being displayable on a user interface such as, but not limited to, a clinician portal.

It will be understood that the term clinician may refer to a device therapy technician, sleep study technician, a physician, or other medical worker (e.g. health care professional) suitably experienced to perform (or assist with) the example methods and systems of patient management and care of the present disclosure.

In some instances, the example methods and/or systems may comprise displaying at least some patient management information and tools (and/or device management information and tools) via a user interface (e.g. graphical user interface), such as on a desktop workstation, mobile computing tablet (or other convenient mobile computing device).

In some examples, one aspect of the example methods and/or systems of patient management may comprise a patient remote control communicating its usage information wirelessly via a patient app (e.g. on a mobile device) to a clinician portal. Via such arrangements, the clinician may receive patient usage information very quickly, and in a manner convenient to the patient because the usage information may be sent from the patient's home, etc. The clinician may receive this usage information after each night's use of the stimulation therapy such that, with regard to at least nightly usage metrics, the clinician is kept up to date on a near real-time basis.

This arrangement stands in sharp contrast to some commercially available systems which may communicate usage information from a patient remote control by requiring the patient to bring their patient remote control to a facility at which the usage information may be downloaded, transferred, etc. from the patient remote control to the clinician computing device for viewing on a clinician portal. In such commercially available arrangements, a clinician would receive updates regarding patient usage considerably less frequently and less conveniently than the nightly usage data available each day in the various examples of the present disclosure.

At least some examples of the present disclosure provide tools to obtain information about how and when a patient volitionally utilizes stimulation therapy based on the times and days that the patient turns the therapy on and off. This information provides objectivity regarding when a patient starts, pauses, and/or ends nightly therapy, among other information such as stimulation amplitude changes, and the like. In some examples, this objective information may be displayed in a user interface (viewable by a clinician) together with subjective information, such as a patient's subjective experience during nightly stimulation therapy, after such therapy (e.g. during the daytime), and the like. By providing both the objective information and the subjective information juxtaposed (or otherwise located nearby) in a user interface, a clinician may enhance their ability to discern relationships between stimulation therapy settings, volitional patient usage (or non-usage) of the stimulation therapy, patient symptoms, and the like. The discernment of such relationships may, in turn, inform decisions resulting in changes to stimulation therapy parameters or resulting in no change to stimulation therapy parameters, among other considerations.

Accordingly, via at least some of these examples, a clinician may efficiently and effectively become informed of the status, progress, health of their patients, which may contribute to patient adherence, therapy efficacy, and improved patient outcomes. By providing patient stimulation therapy usage information on such a timely basis, the example patient management systems and methods also may facilitate a clinician in making interventions, patient education/encouragement, etc. within a time frame more likely to maintain or improve a trajectory of successful patient adherence, therapy efficacy, and/or patient outcomes. At least some example interventions include adjusting stimulation therapy settings and then application of the modified stimulation settings during delivery of stimulation therapy to the patient (via the IMD) during a nightly therapy period.

Moreover, in some such examples, while the example display tools (e.g. available within a user interface) may inform the clinician in evaluating stimulation therapy treatment decisions, the example display tools still provide the clinician with appropriate autonomy in making medical decisions and/or using their discretion as appropriate regarding adjustment of parameters of the programmer, IMD, etc.

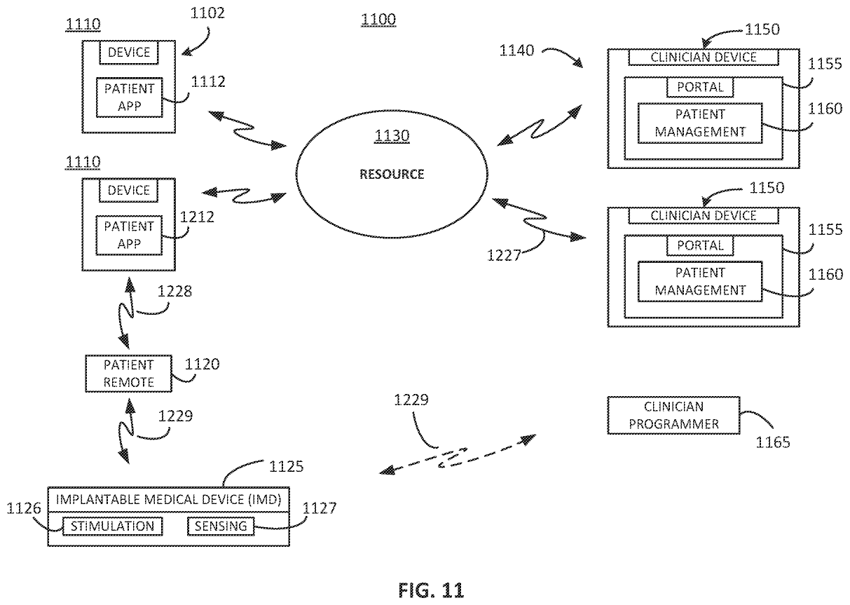

These examples, and additional examples, are described in association with at least FIGS. 11-17B.

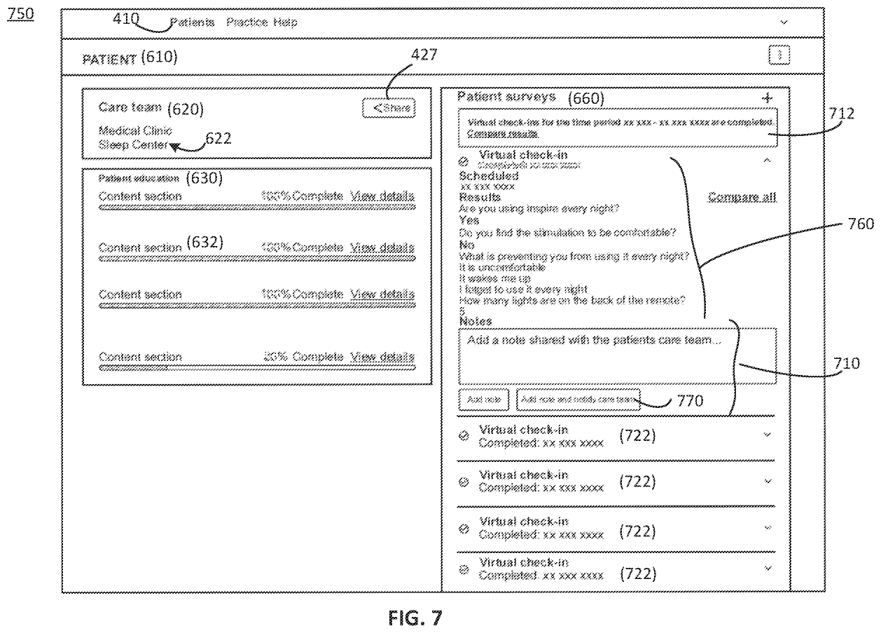

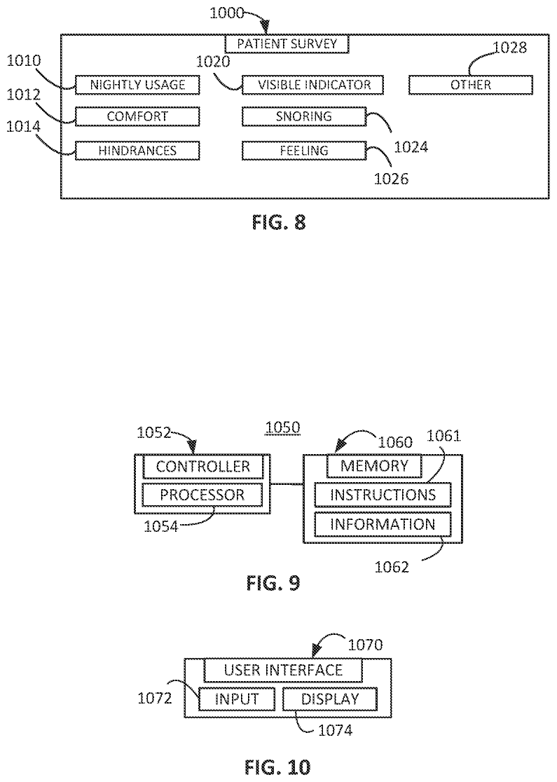

FIG. 1 is a block diagram schematically representing an example arrangement 200, deployable in a method or as a system, to solicit, manage, sort, and communicate patient information to facilitate patient care. As shown in FIG. 1, example arrangement 200 may comprise an array 202 of computing devices 210, each of which host a patient app 212 relating to patient care. The patient app 212 may provide patient education, enable communication with a caregiver, device servicer, device manufacturer, etc. In some such examples, at least some of the computing devices 210 comprise a mobile computing device, such as a mobile phone, tablet, smartwatch, etc. which has a user interface (e.g. 1070 in FIG. 10) to provide for operation of, and display of, the patient app 212. As further shown in FIG. 1, in some examples the patient app 212 may comprise a virtual check-in app or function 220 to facilitate a patient checking in with a caregiver, such as a clinician. Among other functions and features, the virtual check-in app 220 may comprise a patient survey 223, which leads the patient through a series of questions (i.e. queries) regarding their use of their therapy device, how often they use the device, what may be preventing them from using the device, and the like. At least some example queries are later described further in association with at least FIGS. 7 and 8.

As further shown in FIG. 1, the devices 210 may communicate with other devices, entities, etc. via cloud 230 via a wireless communication protocol as represented by directional arrows 227 and/or wired communication protocol in some examples. It will be understood that cloud 230 may comprise a computing resource, including programming, provided via a third party to provide, support, and manage the patient management app for the care entities 250A-250C and the patient app 212 for the patients, which facilitates patient care and facilitates coordinated interaction among the care entities 250A-250C with each other and the respective patients. In some examples, cloud 230 may comprise at least a portion or, and/or an example implementation of, the control portion 1050 (FIG. 9) and user interface 1070 FIG. 10). In some examples, the cloud 230 may be provided via a device manufacturer or servicer, or third party contracted by a device manufacturer. The cloud 230 may be hosted via the internet, world wide web, and/or other network communication link.

As further shown in FIG. 1, example arrangement 200 may comprise an array 240 of entities 250A, 250B, 250C, which provide care in some manner to a patient associated with one of the devices 210. The entities 250A, 250B, 250C may work together in at least some aspects to help coordinate care for the patient(s). Each entity may provide a particular form of expertise in patient care, such examples in which one entity 250A comprises a medical clinic, another entity 250B comprises a sleep center, and other entities supportive of patient care 250C. There may greater or fewer than three entities which form at least part of a care team.

Each entity 250A-250C comprises a computing resource, such as workstation or other computing device which may be stationary or mobile, including a user interface (e.g. 1070 in FIG. 10) to support operation and display of a clinician portal 260. Among other functions and features, the clinician portal 260 may comprise a patient management app 270 by which the particular care provider (e.g. medical clinic, sleep center, etc.) may manage patient care among a group of patients, for an individual patient, etc. The patient management app 270 also may enable communicating with other entities (e.g. among 250A-250C) regarding patient care of the patients associated with devices 210. In some examples, the entities 250A-250C may communicate with each other via cloud 230 (e.g. network communication link, internet, web, etc.) as represented via indicators 227.

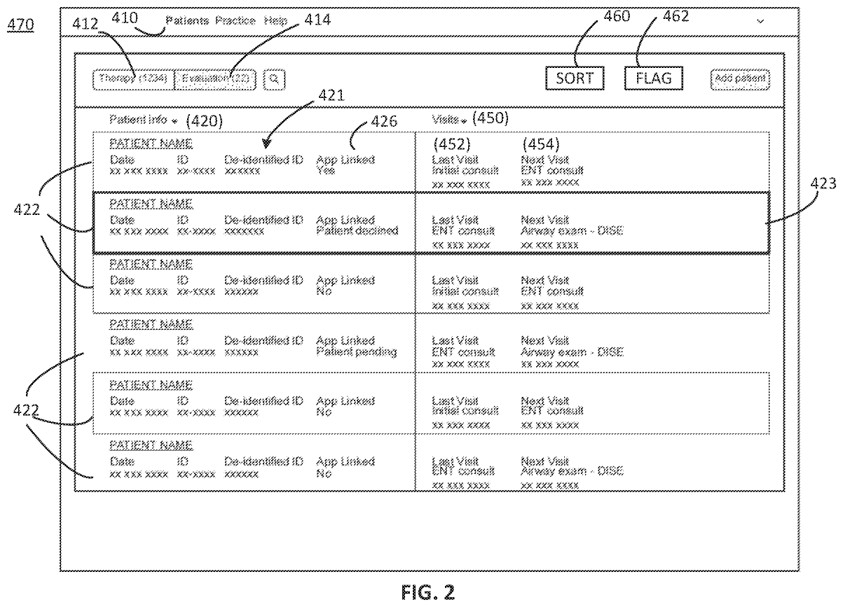

At least some features and attributes of the patient management app 270 and/or patient app 212 are further described in association with FIGS. 2-10.

FIGS. 2-7 are diagrams schematically representing several example user interfaces, such as graphical user interfaces, displayable on a display screen associated with a computing device which may be mobile (e.g. phone, tablet, etc.) or stationary (e.g., desktop, workstation, etc.). In some examples, the user interfaces in FIGS. 2-7 may comprise example implementations of the patient management app 260 operable and displayable as a user interface on clinician portal 260, as described in association with FIG. 1. Accordingly, these example user interfaces in FIGS. 2-7 also comprise example implementations of the control portion 1050 in FIG. 9 and/or user interface 1070 in FIG. 10.

It will be further understood that the arrangement of the various portions displayed on each respective example user interface and/or the detailed listings within each respective displayed portion may act as a workflow method which a clinician (and/or a patient in some instances) may perform to advance patient care. Similarly, it will be apparent from FIG. 1 and the user interfaces in FIGS. 2-7, and the engines, control portions, etc. in association with FIGS. 8-10, that the various features, arrangements, components, etc. may be embodied as a system or apparatus.

FIG. 2 is a block diagram schematically representing an example user interface 400 for patient care, and which may comprise one example implementation of the patient management app 260 (FIG. 1), control portion 1050 (FIG. 9), and/or user interface 1070 (FIG. 10). As shown in FIG. 2, the user interface 400 comprises a banner including a patient function selector 410, among other function selectors (e.g. practice, help, etc.). With the patient function selected, one example user interface 400 comprises a therapy user interface function 412 and an evaluation user interface function 414. FIG. 2 represents an example implementation upon selection of the evaluation function 414, which includes patient information 420 and visit information 450 for a group 421 of example patient listings 422 (e.g. patient record). Each patient listing 422 comprises, under patient information 420, a name, date, ID, de-identified ID, and whether or not the patient has linked (426) via the app to the patient management app 260 (at care entities 250A-250C) and the cloud 230. It will be further understood that each patient listing 422 may comprise visit information 450, such as a date and type (e.g. Initial consult, ENT consult, etc.) of a Last Visit 452 and/or a date and type (e.g. ENT consult, Airway exam, etc. of a Next Visit 454. It will be understood, of course, that for the many different types of patient care which may be provided, the types of visit may vary greatly and accordingly the types of visits (e.g. ENT consult, Airway exam) shown here are merely examples and are not limiting.

As further shown in FIG. 2 via the bolded box 423 outlining the second patient listing, a caregiver (e.g. clinician) may select a particular patient for further review, with at least some of those details being illustrated and described in association with FIGS. 2-8.

As further shown in FIG. 2, the user interface 400 may comprise a sort function 460 and/or a flag function 462. In some examples, the sort function 460 may enable a clinician to sort the patient listings 422 according to various criteria, including which types of visits they had and/or the date on which the visit occurred. In some examples, the sort function 460 may be implemented automatically by which the user interface 400 automatically sorts the patient listings according to criteria pre-selected by the clinician and/or device manufacturer, etc. As a result, upon the clinician opening the user interface 400, the patient listings 422 may already be sorted according to the clinicians preferred criteria. Among other features, this arrangement may help the clinician quickly determine which patients the clinician would like to assess first or last, etc.

In some examples, the sort function 460 may perform its sorting according to which patient listings are flagged (per flag function 462), with the sort function 460 also having selection capabilities for which flags are to be sorted, in some examples.

As further shown in FIG. 2, the user interface 400 may comprise a flag function 462, which may enable a clinician to quick spot patient listings 422 which may warrant faster or more attention. The flag function 462 may comprise a manual selection feature by which the clinician may select a patient listing 422 to be flagged and/or may comprise an automatic selection feature by which the patient listings are automatically flagged according criteria pre-selected by the clinician and/or device manufacturer, etc. As a result, upon the clinician opening the user interface 400, at least some of the patient listings 422 may already be flagged according to the clinicians preferred criteria based on the flag function 462. Among other features, this arrangement may help the clinician quickly determine which patients the clinician would like to assess first or last, etc.

Among other criteria by which a patient listing may be sorted and/or flagged, the user interface 400 may enable sorting and/or flagging patient listings according to those patients which have missed appointments, are no longer seeking care, as well as other parameters further addressed in later Figures, such as the patient stopping use of the device, underutilization, discomfort, and the like.

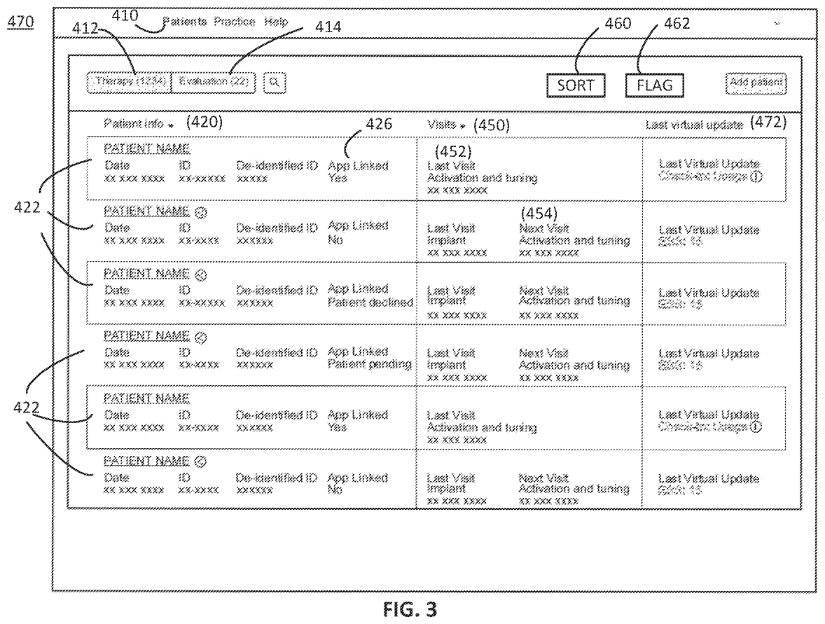

FIG. 3 is a block diagram schematically representing an example user interface 470, which comprises at least some of substantially the same features and attributes of the user interface 400 of FIG. 2, except being an example implementation of selection of the therapy function 412 (instead of the evaluation function 414). As shown in FIG. 3, user interface 470 comprises the same type of patient listings 422 regarding patient information 420 and visit information 450, except with the last visit 452 and next visit 454 information pertaining to therapy-related milestones instead of the evaluation-related milestones shown in FIG. 2. As shown in FIG. 3, in some examples at least some therapy-related milestones may be related to treating sleep disordered breathing (SDB), such “implant” and “activation and tuning”.

As further shown in FIG. 3, user interface 470 may further comprise, for each patient listing, a “last virtual update” indicator 472, by which the clinician will become informed of a therapy effectiveness indication, usage indication, etc. to help the clinician determine which patients are doing well and which may warrant faster or deeper attention. In some such examples, the content of the indicator 472 for each patient listing 422 may be sorted (manually or automatically) per sort function 460 and/or may be flagged (manually or automatically) per flag function 462, in a manner similar to that previously described for user interface 400 in FIG. 2.

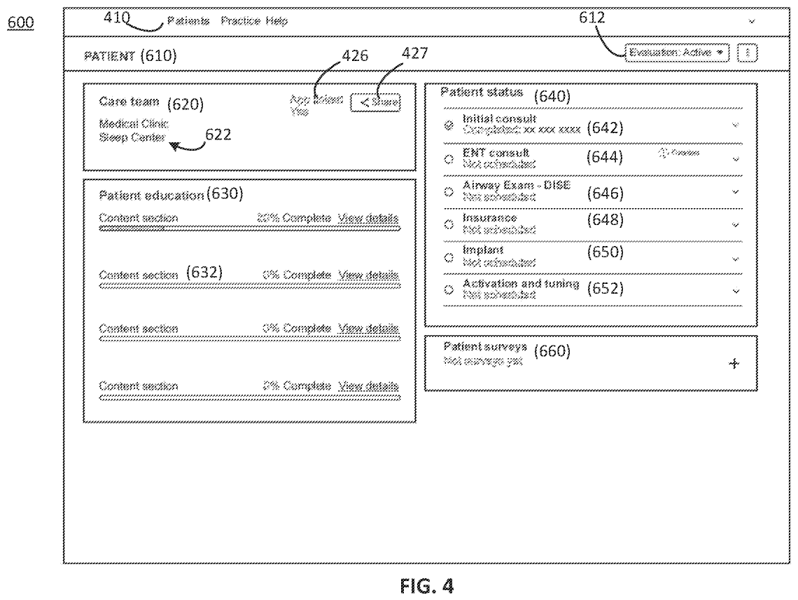

FIG. 4 is a block diagram schematically representing an example user interface 600. As shown in FIG. 4, user interface 600 provides one example implementation of a user interface viewable by a clinician upon selecting, for further review, one of the patient listings 422 in user interfaces 400 (FIG. 2) or 470 (FIG. 3). The particular patient listing 422 may be selected for ordinary review or because the particular patient listing 422 was brought to the immediate attention of the clinician because it was sorted and/or flagged (in the manner previously described) to become conspicuously noticeable to the clinician in viewing one of the user interfaces 400, 470.

As shown in FIG. 4, the user interface 600 comprises a banner 410 with a patient view selector 410, and an individual patient listing 610. In normal usage, the patient's name or other unique identifying information would be listed on user interface 600 at 610 or elsewhere. In some examples, the banner area also may comprise an evaluation indicator 612 to indicate that the patient listing is displaying patient evaluation information, such as but not limited to evaluating whether the patient is suitable for a particular type of therapy. In the case of sleep disordered breathing, the evaluation may be regarding whether the patient is a suitable candidate for upper airway stimulation, CPAP, or other types of therapy. With this in mind, as shown in FIG. 4, in some examples, the user interface 600 may comprise a patient status portion/function 640 which includes a listing of several different evaluation milestones 642-652. Each milestone may correspond to a particular medical procedure, examination, consultation, or relate to other actions such as payment information (e.g. insurance accepted). Each listed milestone 642-652 also may comprise an indication of whether the particular milestone was completed. In one particular example, evaluation milestones relating to care for sleep disordered breathing (SDB) may comprise initial consultation (642), EarNoseThroat (ENT) consultation 644, Airway Exam (Drug Induced Sleep Endoscopy—DISE) 646, Insurance 648, Implant (surgical implantation of a therapy device) 650, and Activation and Tuning (of the implanted device) 652.

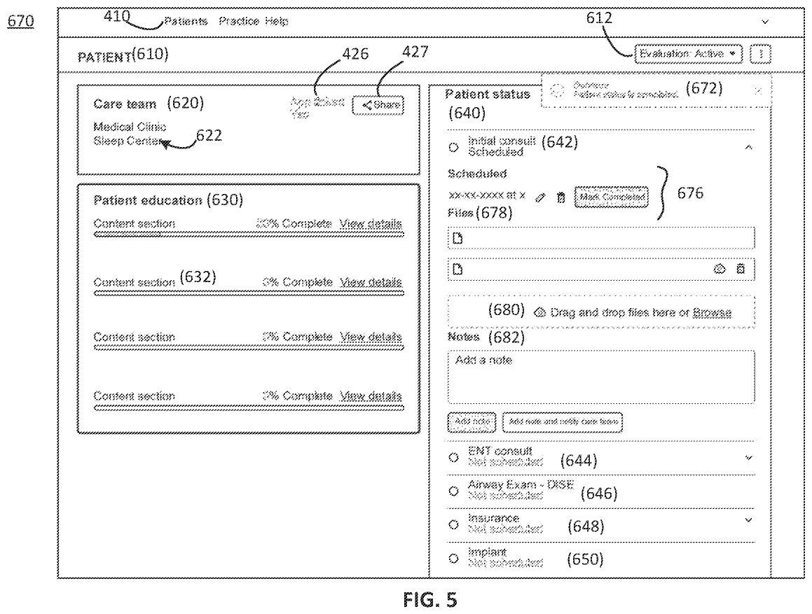

Among other features, the patient status portion 640 of user interface 600 conveniently provides a summary and sequence by which a clinician may immediately recognize which evaluation milestones (642-652) have been met by the patient and which milestones have yet to been achieved. Accordingly, the patient status portion 640 aids the clinician in quickly determining on what path to proceed to help this particular patient, and/or may help the clinician to quickly determine which care team member they should communicate to facilitate progression of the patient through the various milestones. As desired, the clinician may select one of the listed milestones 642-652 to be taken to another user interface screen to obtain more detailed information regarding the selected milestone. As an example, upon selection of the initial consult milestone 642, the clinician will be taken to user interface 670 in FIG. 5, which will be further described later.

In some examples, the patient status portion 640 also may facilitate patient care by the device manufacturer (or servicer) upon that entity becoming aware of certain milestones which a particular patient or a sampling of patients may have trouble achieving, and then take action to facilitate better outcomes in completing such milestones.

However, with further reference to FIG. 4, user interface 600 also comprises a care team portion 620 which provides a listing 622 of the particular care team providers (e.g. entities 250A-250C in FIG. 1) which are caring for this particular patient. Accordingly, via this portion 620, a clinician may quickly determine the members of the care team. In some examples, portion 620 also may comprise an “app linked” function 426 to determine whether the patient and/or a care team member has linked into the cloud 230 (and therefore the patient management system). In some examples, portion 620 also comprises a share function 427 may enable initiation of sharing information with a care team member and/or confirmation of the ability of a care team member to share care information.

As further shown in FIG. 4, in some examples the user interface 600 comprises a patient education portion 630, which displays the progress the particular patient has made in viewing different content education modules 632. In some examples, these content education modules 632 may be provided and displayed via the patient app 212 on patient devices 210, while in some examples they may be viewable via other resources linked to the cloud 230. Data from the patient's engagement with the patient content modules 632 is processed via the cloud 230 and provided for display in patient education portion 630 of the user interface 600. In some examples, the patient education portion 630 may illustrate a degree of progress according to percentage completion based on a percentage and/or a bar-type graphing. Further details can be viewed.

Among other features associated with the patient education portion 630, a clinician may quickly determine whether the patient is making adequate progress in patient education, which in turn, may facilitate the type and manner of care provided by the clinician. In addition, this progress information also may be sorted and/or flagged according to the sort function 460 and/or flag function 462 (previously described in FIGS. 2-3) to help the clinician quickly identify a patient which may need more encouragement to complete their evaluations.

As further shown in FIG. 4, in some examples user interface 600 may comprise a patient survey portion 660, which tracks whether a patient has completed a patient survey as part of a virtual check-in and/or how many (and when) their patient surveys were completed. In some examples, the patient survey portion 660 may indicate that no surveys have been completed, such as when the patient is still at the initial consultation milestone of their evaluation.

FIG. 5 is block diagram schematically representing an example user interface 670 comprising at least some of substantially the same features and attributes as user interface 600 in FIG. 4, except further comprising a more detailed view of the initial consult milestone 642. With this in mind, a clinician may progress from user interface 600 to user interface 670 upon selecting the initial consult milestone 642 in FIG. 4.

As shown in FIG. 5, in some examples the expanded view of the initial consult milestone 642 may comprise scheduling details 676 as shown (date, completed, etc.), related files 678 (e.g. patient assessment notes, images, etc.) file upload 680, and Note portion 682. As further shown, the patient status portion 640 also may show at least some of the other milestones 644-650 below the expanded milestone window 642. In some examples, a pop-up window 672 may appear to provide additional information.

It will be understood that upon selection of one of the other milestones, an expanded window would appear in user interface 600, 670 with details particular to that milestone.

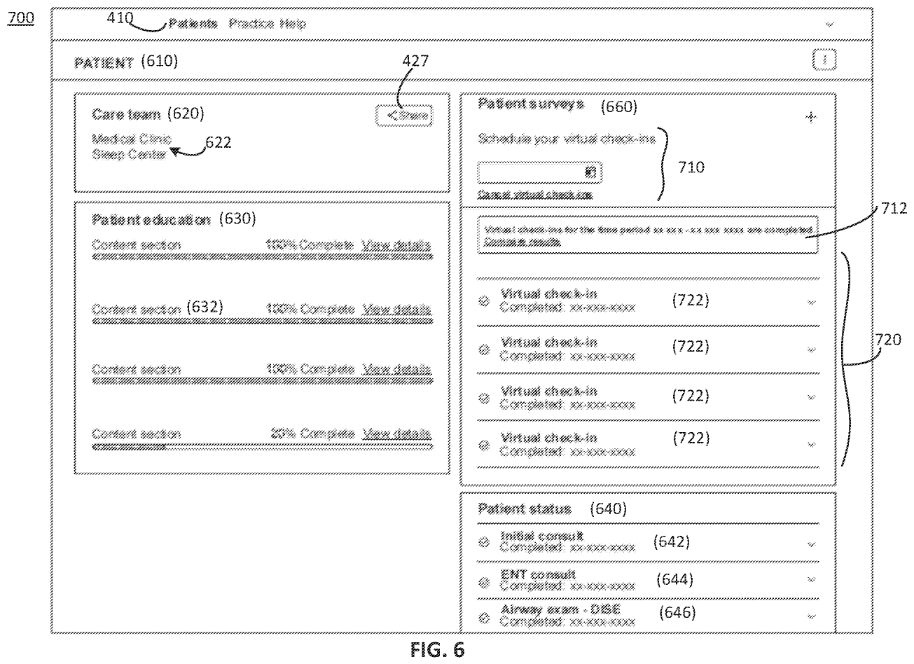

FIG. 6 is a block diagram schematically representing an example user interface 700 comprising at least some of substantially the same features and attributes as user interface 600 in FIG. 4, except further comprising an enlarged patient survey portion 660, which appears more prominently above the patient status portion 640. In some examples, the user interface 700 may be used for a patient which has an implanted therapy device and which has started therapy, such that the patient status portion 640 may have relevance since the patient has already completed all of the evaluation milestones.

As shown in FIG. 6, the patient survey portion 660 comprises a scheduling portion 710 to facilitate scheduling a virtual check-in. Meanwhile, a records access function 712 permits a clinician to access one or more records of past virtual check-ins. As further shown in FIG. 6, the patient survey portion 660 also may comprise a listing 720 of patent virtual check-in records 722, with each listed record 722 including a completion date.

Upon selection of one of the virtual check-in records 722 in listing 720 of user interface 700, the clinician is taken to user interface 750 in FIG. 7, which provides an expanded portion 760 displaying the details of the selected virtual check-in. In some examples, user interface 750 collapses or hides the patient status portion 640 to make room for the expanded portion 760.

As shown at 760 in FIG. 7, in some examples the virtual check-in details include various automated queries and answers supplied by the patient from their engagement of the patient survey 223 on the virtual check-in 220 of the patient app 220 on device 210, as shown in FIG. 1. Moreover, the provided details may include the scheduled date. While the expanded portion 760 within user interface 750 illustrates at least some example detailed queries and answers, a further description regarding such queries and answers as part of a patient survey is provided below in association with at least FIG. 8.

FIG. 8 comprises a block diagram schematically representing an example patient survey engine 1000. In some examples, the example patient survey engine 1000 may comprise a portion of, and/or be implemented via, control portion 1050 in FIG. 9. In general terms, the patient survey engine 1000 controls and supports operation of a patient survey (e.g. 223 in FIG. 1; results 760 in FIG. 7) displayed on a user interface (e.g. 1070 in FIG. 10), such as via an app on a display screen of a computing device, which may be mobile such as a mobile phone, tablet, etc. However, it will be understood an example patient survey may be displayed and provided on a user interface of a stationary computing device, in some examples.

As shown in FIG. 8, in some examples the patient survey engine 1000 may comprise parameters to query a patient regarding: (A) nightly usage (1010) (“are you using the device every night?); (B) comfort (1012) (“is the stimulation comfortable?”); (C) hindrances (1014); (D) device visible indicators (1020); (E) snoring (1024) (“has your snoring improved?”); (F) feeling (1026) (e.g. “do you feel tired?”), and/or other parameters (1028).

In some examples, hindrances 1014 may relate to factors which are preventing (or perceived to be preventing) a patient from employing therapy every night. At least some example hindrance factors may comprise: (1) it is uncomfortable; (2) it wakes me up; (3) I forget to use it every night, and/or (4) other reasons. In some examples, the device visible indicators parameter 1020 may relate to a patient query of what the patient observes regarding visible indicators on a therapy component, such as a patient remote control. The visible indicators may provide an indication of the amplitude strength being used, in some examples. In some examples, other types of therapy or usage parameters which may be conveyed via visible indicators on a housing of the patient remote control or other therapy peripheral.

The results of one example patient survey are illustrated in the previously described, example survey results 760 of user interface 750 in FIG. 7.

It will be understood that such example patient surveys are not limited to the exact words noted above and may use different expression, terms, etc. while still seeking and obtaining the same type of information. Moreover, as evident by the other parameter 1028, the example patient survey provided via patient survey engine 1000 in FIG. 8 is not strictly limited to the example queries shown in FIG. 7 or described above in association with FIG. 8.

FIG. 9 is a block diagram schematically representing an example control portion 1050. In some examples, control portion 1050 provides one example implementation of a control portion forming a part of, implementing, and/or generally managing the example arrangements, including clinician portals, cloud resources, patient management systems, patient mobile apps, user interfaces, control portion, instructions, workflows, engines, functions, parameters, and/or methods, as described throughout examples of the present disclosure in association with FIGS. 1-10. In some examples, control portion 1050 includes a controller 1052 and a memory 1060. In general terms, controller 1052 of control portion 1050 comprises at least one processor 1054 and associated memories. The controller 1052 is electrically couplable to, and in communication with, memory 1060 to generate control signals to direct operation of at least some of the example arrangements, including clinician portals, cloud resources, patient management systems, patient mobile apps, user interfaces, control portion, instructions, workflows, engines, functions, parameters, and/or methods, as described throughout examples of the present disclosure. In some examples, these generated control signals include, but are not limited to, employing instructions 1061 and/or information 1062 stored in memory 1060 to at least direct and manage sleep disordered breathing (SDB) care (e.g. sensing, stimulation, etc.) in the manner described in at least some examples of the present disclosure, such as but not limited to patient care and management including clinician portals, workflows and a patient virtual check-in. In some instances, the controller 1052 or control portion 1050 may sometimes be referred to as being programmed to perform the above-identified actions, functions, etc.

In response to or based upon commands received via a user interface (e.g. user interface 1070 in FIG. 10 or example user interfaces throughout FIGS. 1-8) and/or via machine readable instructions, controller 1052 generates control signals as described above in accordance with at least some of the examples of the present disclosure. In some examples, controller 1052 is embodied in a general purpose computing device while in some examples, controller 1052 is incorporated into or associated with at least some of the example arrangements, including clinician portals, cloud resource, patient management systems, patient mobile apps, user interface, control portion, instructions, workflows, engines, functions, parameters, and/or methods, etc. as described throughout examples of the present disclosure.

For purposes of this application, in reference to the controller 1052, the term “processor” shall mean a presently developed or future developed processor (or processing resources) that executes machine readable instructions contained in a memory or that includes circuitry to perform computations. In some examples, execution of the machine readable instructions, such as those provided via memory 1060 of control portion 1050 cause the processor to perform the above-identified actions, such as operating controller 1052 to implement sleep disordered breathing (SDB) care via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1060. The machine readable instructions may include a sequence of instructions, a processor-executable machine learning model, or the like. In some examples, memory 1060 comprises a computer readable tangible medium providing non-volatile storage of the machine readable instructions executable by a process of controller 1052. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 1052 may be embodied as part of at least one application-specific integrated circuit (ASIC), at least one field-programmable gate array (FPGA), and/or the like. In at least some examples, the controller 1052 is not limited to any specific combination of hardware circuitry and machine readable instructions, nor limited to any particular source for the machine readable instructions executed by the controller 1052.

In some examples, control portion 1050 may be entirely implemented within or by a stand-alone device.

In some examples, the control portion 1050 may be partially implemented in one of the example arrangements, clinician portals, cloud resources, patient management systems, patient mobile apps, etc. and partially implemented in a computing resource separate from, and independent of, the example arrangements, including clinician portals, cloud resources, patient management systems, patient mobile apps, etc. but in communication with such example arrangements, etc. For instance, in some examples control portion 1050 may be implemented via a server accessible via the cloud and/or other network pathways. In some examples, the control portion 1050 may be distributed or apportioned among multiple devices or resources such as among a server, an example arrangement, and/or a user interface.

In some examples, control portion 1050 includes, and/or is in communication with, a user interface 1070 as shown in FIG. 10. In some examples, at least some portions or aspects of the user interface 1070 are provided via a graphical user interface (GUI), and may comprise a display 1074 and input 1072. In some examples, user interface 1070 comprises a user interface or other display that provides for the simultaneous display, activation, and/or operation of at least some of the example arrangements, clinician portals, cloud resources, patient management systems, patient mobile apps, user interface, control portion, workflows, instructions, engines, functions, parameters, and/or methods, etc., as described in association with FIGS. 1-8. For instance, the various user interfaces described in association with FIGS. 1-8 may each provide an example implementation of user interface 1070.

FIG. 11 is a block diagram schematically representing an example arrangement 1100, deployable in an example method of (or as an example system for) patient management to facilitate patient care. As shown in FIG. 11, example arrangement 1100 may comprise an array 1102 of computing devices 1110, each of which host a patient app 1112 relating to patient care. The devices 1110 may sometimes be referred to as patient devices, patient computing devices, and the like. The patient app 1112 may provide patient education and/or enable communication with a caregiver, device servicer, device manufacturer, etc. In some examples, the patient app 1112 may communicate patient usage information (and some related therapy metrics) to a clinician device 1150 (e.g. care entity), device servicer, device manufacturer, etc. In some such examples, at least some of the patient devices 1110 may comprise a mobile computing device, such as a mobile phone, tablet, smartwatch, etc. which has a user interface (e.g. 2640 in FIG. 17B) to provide for operation of, and display of, the patient app 1112.

As shown in FIG. 11, in some examples the example arrangement 1100 may comprise an implantable medical device (IMD) 1125. In some examples, the IMD 1125 may be adapted for treating sleep disordered breathing (SDB) and/or other patient conditions (e.g. cardiac, pelvic disorders, etc.). A patient remote control 1120 may communicate with the IMD 1125 via a wireless communication protocol 1229 either directly or indirectly via an intermediary communication element (e.g. antenna, other). In some examples, such wireless communication may take the form of inductive telemetry. In some examples, the IMD 1125 may comprise an implantable pulse generator (IPG) for generating stimulation therapy signals to be delivered to the patient via a stimulation element (e.g. electrode) within the patient.

In general terms, the patient remote control 1120 enables a patient to have limited control over their stimulation therapy, such as turning the stimulation therapy on/off, pause, and/or increasing or decreasing the amplitude of stimulation within a lower and upper limit set by a clinician (and/or device manufacturer, supplier, etc.). In some examples, the patient remote control 1120 also tracks patient usage of these controls to enable a clinician, the patient, and others to learn about the patient's usage, therapy effectiveness, patient adherence, etc. In some examples, the patient remote control also may receive some information from the IMD 1125 regarding stimulation metrics, sensing metrics, etc.

In some examples, the patient remote control 1120 is in communication with the patient app 1112 such that patient app 1112 on device 1110 may receive the patient usage information from the patient remote control 1120, as well as whatever therapy, sensing, etc. information was communicated from the IMD 1125 to the patient remote control 1120. In some examples, the communication between the patient remote control 1120 and the patient app 1112 (on patient device 1110) may occur wirelessly 1228 via a number of wireless communication protocols such as, but not limited to, a Bluetooth® wireless communication protocol. In some examples, the communication between patient remote control 1120 and patient app 1112 (on patient device 1110) may occur via a wired connection.

As noted here and elsewhere, the patient app 1112 may communicate this information (received from the patient remote control 1120) to one or more of the clinician devices 1150 via resource 1130 to facilitate patient management according to examples of the present disclosure. In some examples, the patient app 1112 also may obtain some patient information through the patient's use of the app 1112 which also may be communicated to the clinician devices 1150 separately from, or integrated with, the patient usage information and therapy information from the patient remote control 1120 and/or IMD 1125.

As further shown in FIG. 11, an example IMD 1125 may comprise a stimulation component 1126 and/or sensing component 1142. In some examples, the stimulation component 1126 comprises a stimulation engine to generate a stimulation signal to be applied to a tissue (e.g., nerve, muscle, etc.). In the examples in which the IMD 1125 comprises an implantable pulse generator (IPG), the tissue to be stimulated may comprise tissue to maintain or restore upper airway patency, such as but not limited to a hypoglossal nerve, an ansa cervicalis-related nerve, and/or phrenic nerve, etc. In some such examples, the stimulation component 1126 also may comprise circuitry for generating and delivering the stimulation signal. In some examples, the stimulation component 1126 of the IMD 1125 also may comprise a stimulation element, such as an electrode through which the stimulation signal may be applied to the target tissue.

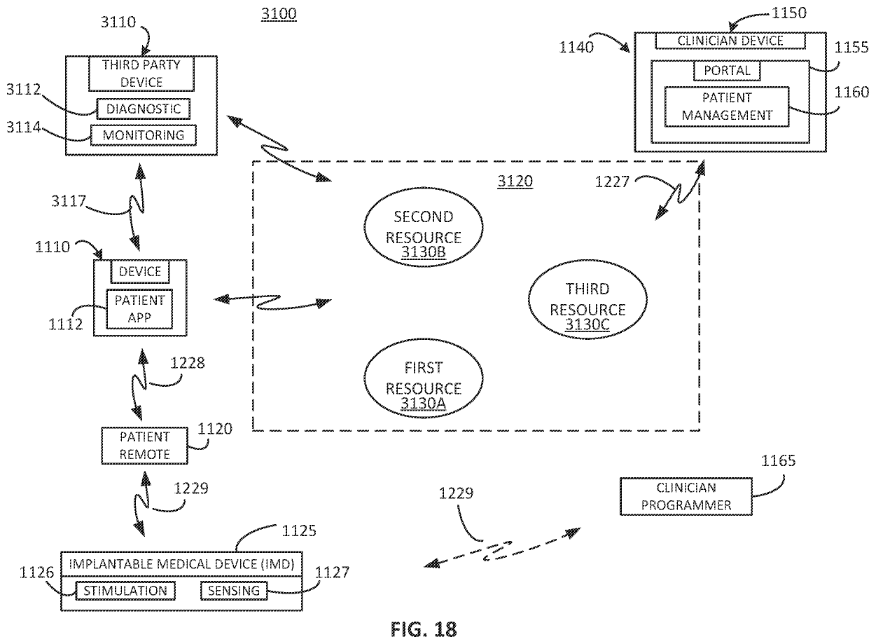

In some examples, the sensing component 1127 comprises a sensing engine to receive a sensing signal obtained relative to a tissue (e.g., muscle, organ, etc.). In the examples in which the IMD 1125 comprises an IPG for treating sleep disordered breathing (SDB), the tissue to be sensed may be related to respiration, oxygenation, cardiac functions, upper airway patency, and the like. In some such examples, the sensing component 1127 also may comprise circuitry for receiving and processing the sensing signal. In some examples, the sensing component 1127 of the IMD 1125 also may comprise a sensing element, such as an electrode or other element through which the sensing signal is obtained. In some examples, the sensing element may comprise an accelerometer for determining sleep information, respiratory information, posture information, physical control information. The accelerometer may be implantable, and in some examples, may be incorporated within a device including a stimulation generating element (e.g. 1126), such as an implantable pulse generator, which may comprise one example implementation of the implantable medical device 1126 in FIG. 18.

Of course, in contexts in which the IMD 1125 relates to bodily organs, functions, etc. other than sleep disordered breathing, the stimulation component 1126 and sensing component 1127 would be deployed relative to other tissues. For instance, the IMD 1125 may be deployed to treat pelvic disorders, such as stress incontinence or other conditions, with applicable tissues including the bladder, pudendal nerve, urinary and/or anal sphincters and the like.

In some examples, the stimulation component 1126 and/or sensing component 1127 may be on-board the IMD 1125, which in some examples may comprise a microstimulator.

In some examples, at least a portion of the stimulation component 1126 and/or sensing component 1127 may be separate from, and independent of, a housing of the IMD 1125 with one or both components 1126, 1127 being in wired or wireless communication with the IMD 1125.

As further shown in FIG. 11, the patient devices 1110 may communicate with other devices, entities, etc. via resource 1130 via a wireless communication protocol as represented by directional arrows 1227 and/or wired communication protocol in some examples. It will be understood that resource 1130 may comprise a computing resource (including stored programming) provided via a third party to help provide and support patient management with the clinician devices 1150 (e.g. clinician entities, care entities) and the patient app 1112. In some examples, resource 1130 may comprise at least a portion or, and/or an example implementation of, the control portion 2600 (FIG. 17A) and user interface 2640 FIG. 17B). In some examples, the third party providing resource 1130 may comprise a device manufacturer, device supplier, or third party contracted by a device manufacturer. The resource 1130 may be hosted via the internet, World Wide Web, and/or other network communication link.

As further shown in FIG. 11, example arrangement 1100 may comprise an array 1140 of clinician devices 1150, which provide care in some manner to a patient associated with one of the devices 1110. The entities associated with each clinician device 1150 may work together in at least some aspects to help coordinate care for the patient(s). Each entity may provide a particular form of expertise in patient care, such examples in which one entity (associated with a clinician device 1150) may comprise a medical clinic, while another entity (associated with a different clinician device 1150) may comprise a sleep center, and other entities may comprise providers which support patient care in some manner. There may greater or fewer than the two clinician devices (e.g. care entities) shown in FIG. 11 which form at least part of a care team.

Each clinician device 1150 comprises a computing resource, such as workstation or other computing device which may be stationary or mobile, including a user interface (e.g. 2600 in FIG. 17B) to support operation and display of a portal 1155 (e.g. a clinician portal). Among other functions and features, the portal 1155 may comprise a patient management app 1160 by which the particular care provider (e.g. medical clinic, sleep center, etc.) may manage patient care among a group of patients, for an individual patient, etc. The patient management app 1160 also may enable communicating with other entities (e.g. 1150) regarding patient care of the patients associated with devices 1110. In some examples, the clinician devices 1150 may communicate with each other via at least resource 1130 (e.g. network communication link, internet, web, etc.) as represented via indicators 1227.

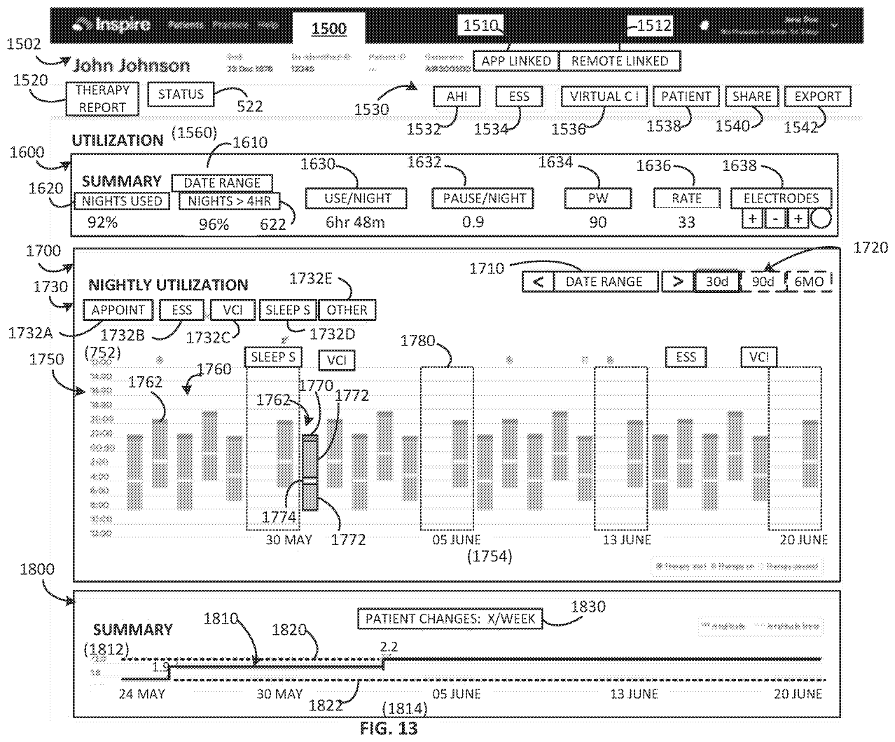

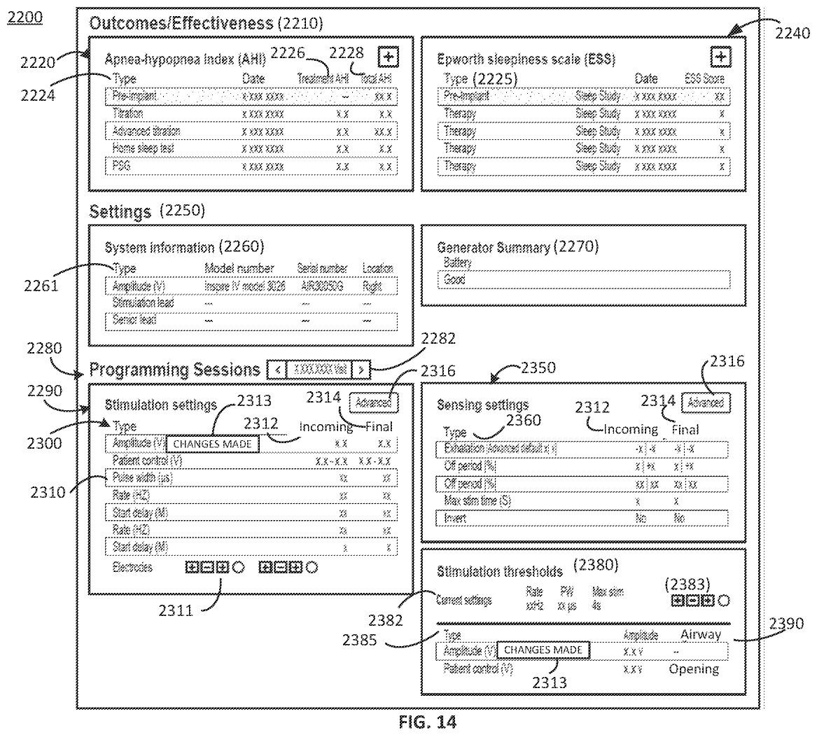

The later-described FIGS. 13-14 are diagrams schematically representing several example user interfaces, such as graphical user interfaces, displayable on a display screen associated with a computing device which may be mobile (e.g. phone, tablet, etc.) or stationary (e.g., desktop, workstation, etc.). In some examples, the user interfaces in FIGS. 13-14 may comprise example implementations of the patient management app 1160 operable and displayable as a user interface on clinician portal 1155, as described in association with FIG. 11. Accordingly, these example user interfaces in FIGS. 13-14 also comprise example implementations of the control portion 2600 in FIG. 17A and/or user interface 2640 in FIG. 17B.

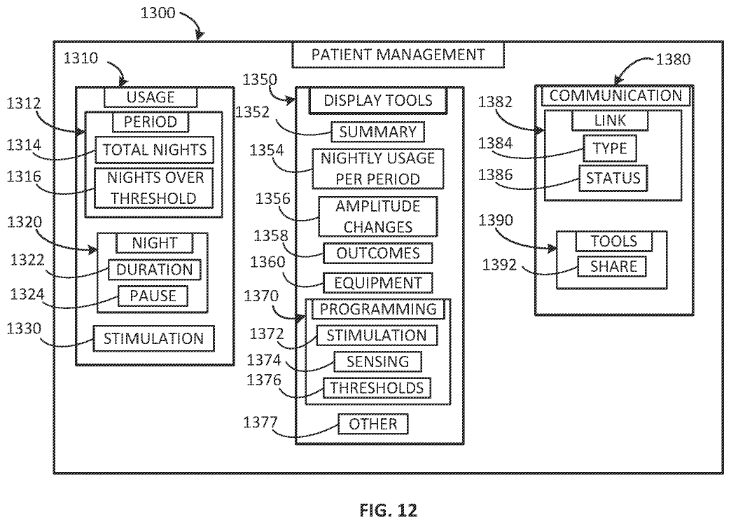

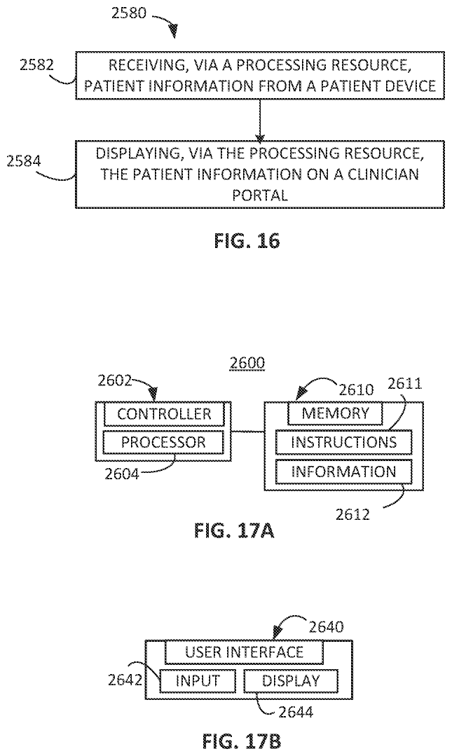

It will be further understood that the arrangement of the various portions displayed on each respective example user interface and/or the detailed listings within each respective displayed portion may form part of a method of patient management (including but not limited to stimulation therapy treatment) which a clinician (and/or other health care professional) may perform to advance patient care. One example method is described later in association with FIG. 16. Similarly, it will be apparent from FIG. 11 and the user interfaces in FIGS. 13-14, and the engines, control portions, etc. in association with FIGS. 12-17A, 17B, that the various features, arrangements, components, etc. may be embodied as a system or apparatus.

In some examples, the example arrangement 1100 may comprise a clinician programmer 1165, which may periodically communicate with the IMD 1125 wirelessly (e.g. inductive telemetry) to initially configure and/or modify the configured stimulation therapy settings, sensing settings, etc. of the IMD 1125.

In some examples, the programmer 1165 comprises a user interface, such as but not limited to, a graphical user interface (GUI) to facilitate display and input relative to workflows by which a clinician operates the programmer 1165. It will be further understood that the programmer 1102 may perform tasks or operations (relating to patient care, maintenance) etc. other than programming stimulation-related aspects of the IMD 1125. Moreover, in some examples, the programmer 1165 may comprise a device dedicated solely for the purpose of communicating with, programming, etc. the IMD 1125. Of course, in some examples, the clinician programmer 1165 also may communicate directly with the clinician portal 1155 (such as via patient management app 1160) to update the patient management system/method regarding at least stimulation settings, etc. which were configured in the IMD 1125 per workflows of the clinician programmer 1165.

However, in some examples, the programmer 1165 may comprise a non-dedicated device which may be used for purposes (e.g. general communication, general computing, etc.) other than communicating with or, programming the IMD 1125. In some such examples, the programmer 1165 may comprise a consumer device, such as a consumer tablet, smart phone, etc. which is also operable via secure modes/communications/paths to communicate with, program, etc. the IMD 1125.

The user interface available on the clinician programmer 1165 may comprise one example implementation of, and/or comprise at least some of the features and attributes of, the user interface 2640 described later in association with at least FIGS. 17A-17B.

Accordingly, it will be further understood that the programmer 1165 and/or IMD 1125 may comprise a control portion, or comprise an example implementation of one part of a control portion, such as control portion 2600 as later described in association with at least FIG. 17A.

As shown in FIG. 12, in some examples a patient management engine 1300 may control, direct, etc. obtaining patient information and displaying such information to help manage patient care, etc. At least some aspects of patient management includes actions, tools, etc. to deliver stimulation therapy to a tissue of a patient, such as but not limited to tissue which may maintain or restore upper airway patency to treat sleep disordered breathing (SDB). In some examples, the example methods and/or systems of the present disclosure directed to patient management may lead to adjustments in stimulation programming (and related settings), which are used to configure the IMD 1125 such that the stimulation programming settings become stored within, and are implemented via the IMD 1125 to deliver stimulation therapy and related care to the patient.

With further reference to FIG. 12, in some examples the patient management engine 1300 comprises a usage tracking engine 1310, a display tools engine 1350, and a communication engine 1380. In some examples, all or some of the features of the patient management engine 1300 may be implemented via, and/or as part of, control portion 2600 as later described in FIG. 17A. Moreover, it will be understood that features and aspects of the patient management engine 1300 are not strictly limited to the engines 1310, 1350, and/or 1380.

At least some aspects of the patient management engine 1300 will be described in association with the user interface 1500 of FIG. 13 and/or the user interface 2200 of FIG. 14. Accordingly, at least some more general aspects of user interface 1500 (FIG. 13) and user interface 2200 (FIG. 14) will be described prior to a more detailed description of various aspects of patient management engine 1300 in association with FIG. 12. It will be further understood that, in at least some examples, the user interface 1500 in FIG. 13 and the user interface 2200 in FIG. 14 may together form a single user interface, or that at least some portions of user interface 1500 (FIG. 13) may appear together, with at least some portions of user interface 2200 (FIG. 14), as a single user interface, such as on a single display screen.

As shown in FIG. 13, the user interface 1500 comprises a general portion 1502 and utilization portion 1560, which in turn includes summary portion 1620, nightly utilization portion 1700, and amplitude changes portion 1800.

In some examples, the general portion 1502 may display information such as patient name, patient ID number(s), equipment identification, and the like. In addition, in some examples, the general portion 1502 may comprise indicators which may act as a function to be activated and/or as a status indicator. For instance, the general portion 1502 may comprise a therapy report indicator 1520, a status indicator 1522, an app linked indicator 1510, a remote linked indicator 1512. For instance, indicator 1510 may indicate whether the particular patient's app 1112 (on patient device 1110) is linked (1510) into the arrangement 1100 (FIG. 11), and particularly relative to the clinician portal 1155 (FIG. 11) at which the user interface 1500 of the patient management app 1160 is being viewed. The remote linked indicator 1512 provides a similar indication regarding the patient remote 1120 (FIG. 11), except regarding whether the particular patient's remote control 1120 (FIG. 11) is linked relative to the patient app 1112 (on a patient device 1110) and/or relative to the IMD 1125 within the patient.

As further shown in FIG. 13, in some examples the general portion 1502 of patient management user interface 1500 may comprise an array 1530 of additional indicators regarding Apnea-Hypopnea Index (AHI) 1532, Epworth Sleepiness Scale (ESS) 1534, virtual check-in (VIRTUAL C I) 1536, patient 1538, share 1540, and export 1542. In some such examples, upon activation via user interface 1500, at least some of the indicators of array 1530 may cause certain information, status, etc. specific to each respective indicator 1532-1542 to become displayed on user interface 1500 or to become dormant on user interface 1500. For example, as previously noted, portions of user interface 2200 in FIG. 14 may sometimes be displayed together with portions of user interface 1500 in FIG. 13, and vice versa. Accordingly, in one non-limiting example, activation of AHI indicator 1532 and ESS indicator 1534 may cause display of AHI information and ESS information for the patient, such as shown in user interface 2200 in FIG. 14. Similarly, activation of virtual check-in indicator 1536 may cause display (and operable access) of certain patient information related to a patient virtual check-in tool. The share indicator 1540 cause selectable patient information to be shared from one clinician device 1150 (e.g. health care personnel at a sleep center) to another clinician device 1150 (e.g. health care personnel at a medical clinic) and the like. The export indicator 1542 enables exporting patient management data.

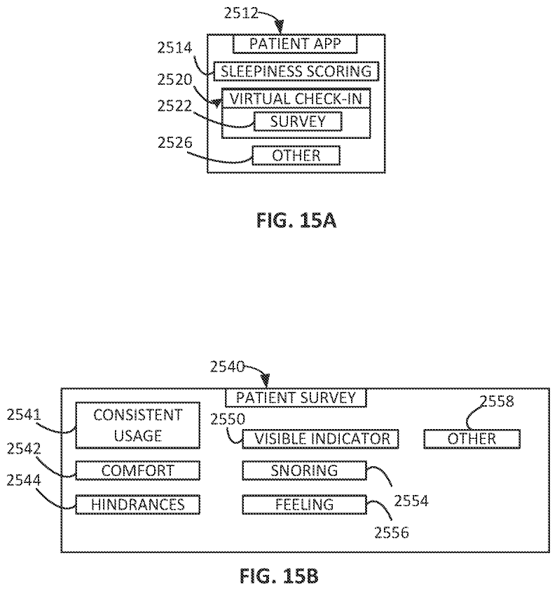

At least some aspects regarding at least some of the above-noted indicators 1532-1542, such as (but not limited to) the virtual check-in (Virtual C I) indicator 1536, are further described later in association with at least FIGS. 15A-15B.

Meanwhile, as shown in FIG. 14, user interface 2200 generally comprises an outcomes/effectiveness (i.e. performance) portion 2210, a setting portion 2250, and a programming portion 2280, which may in turn comprise a stimulation settings portion 2290, a sensing settings portion 2350, and a stimulation thresholds portion 2380.

With these general aspects of the user interfaces 1500 (FIG. 13), 2200 (FIG. 14) in mind, further reference is made to the patient management engine 1300 in FIG. 12, which drives the availability of and operation of the respective user interfaces 1500 (FIG. 13), 2200 (FIG. 14).

With reference to FIG. 13, in some examples the usage engine 1310 of patient management engine 1300 (FIG. 12) may control, direct and/or support a clinician to perform patient management according to (at least) patient usage of a stimulation therapy device, such as IMD 125 (FIG. 11). As further shown in FIG. 12, the usage engine 1310 comprises a period function 1312 by which usage may be tracked according to selectable, predetermined periods of time. Some example periods may comprise a week, a month (e.g. 30 days), a quarter (e.g. 90 days), a half year (e.g. 180 days), and the like. Moreover, in some examples, the period function 1312 may operate according to a total nights parameter 1314 and/or a nights over threshold parameter 1316. In particular, per the total nights parameter 1314, usage engine 1310 may cause tracking of the total nights which the patient used the stimulation therapy device within the selected period (e.g. 30 days). This total may be expressed as a percentage, such as the number of days used relative to the total number of days within the selected period. One example implementation of the total nights parameter 1314 is displayed as parameter 1620 (of summary portion 1600) in the user interface 1500 in FIG. 13, which shows a non-limiting example 92 percent of the stimulation therapy being used during the selected period (e.g. 30 days). In other words, the stimulation therapy was used 92 percent of the nights within the selected period.

With further reference to FIG. 12, per the nights over threshold parameter 1316 of usage engine 1310, the patient management engine 1300 causes tracking the number of nights (within the selected period, e.g. 30 days) for which patient usage of the stimulation therapy met or exceeded a threshold. In some examples, the nights over threshold parameter 1316 may be expressed as nights relative to a threshold or nights relative to a criteria (e.g. not less than “x” hours per night). In some non-limiting examples, the threshold may comprise a selectable time period, such as a number of hours (e.g. 4 hours, 5 hours, and the like). This time period also may be selected as a partial hour metric, such as 3.5 hours, 3.75, 4.5 hours. In some such examples, the selected period of 4 hours may be a significant indicator of a quantity of usage which may result in significantly improved outcomes/effectiveness for the patient regarding their sleep disordered breathing (SDB). In some examples, it will be understood that the threshold may comprise “about” a selectable quantity (e.g. about 4 hours), which includes quantities of 3.8 hours, 3.85 hours, 3.9 hours, 3.95 hours, 4.05 hours, 4.1 hours, 4.15 hours, 4.2 hours, and the like, as an example.

One example implementation of the nights over threshold parameter 1316 is displayed as parameter 1622 (of summary portion 1620) in the user interface 1500 in FIG. 13, which shows a non-limiting example in which, for the days which therapy was used, 96 percent of those days included the patient using stimulation therapy greater than a selected time period of 4 hours.

While each parameter 1314 or parameter 1316 alone may provide significant information regarding patient adherence, together parameters 1314 and 1316 provide a more comprehensive picture of how well and how often the patient is using the stimulation therapy via the IMD 1125.

As further shown in FIG. 12, the usage engine 1310 comprises a night function 1320 by which usage may be tracked, evaluated, etc. according to the details of each night within the selected period (of function 1312). Moreover, in some examples, the night function 1314 may operate according to a duration parameter 1322 and/or a pause parameter 1324. In particular, per the duration parameter 1322, usage engine 1310 may cause tracking of a duration of usage per night, which may be expressed as an average, mean, and the like. One example implementation of the duration parameter 1322 is displayed as parameter 1630 (of summary portion 1600) in the example user interface 1500 in FIG. 13, which shows a non-limiting example in which, for the days which therapy was used, the average duration of stimulation therapy was 6 hours and 48 minutes.

Moreover, per the above-noted pause parameter 1324, usage engine 1310 may cause tracking of the number of times per night which stimulation therapy was paused by the patient, with the number of times being expressed as an average, mean, and the like. One example implementation of the pause parameter 1324 is displayed as parameter 1632 (of summary portion 1620) in the example user interface 1500 in FIG. 13, which shows a non-limiting example in which, for the days which therapy was used, the average number of pauses per night was 0.9.

While either one of the parameter 1322 or parameter 1324 (of night function 1320 in FIG. 12) alone may provide significant information regarding patient adherence, when considered together the parameters 1322 and 1324 may provide a more comprehensive picture of the total amount of stimulation therapy which the patient is receiving each night within the selected period (e.g. 30 day) for the nights therapy was actually used.

As further shown in FIG. 12, the usage engine 1310 also may comprise a stimulation parameter 1330 to track and/or display the stimulation settings for the IMD 1125 which were used to provide the stimulation therapy delivered to the patient. One example implementation of the stimulation settings parameter 1330 is displayed via parameters 1634, 1636, 1638 of summary portion 1600 in the example user interface 1500 in FIG. 13, which shows a non-limiting example in which a pulse width 1634 was 90 microseconds, a pulse rate 1636 was 33 Hertz, and certain electrodes 1638 were used to apply the stimulation therapy.

With further reference to FIG. 12, the patient management engine 1300 comprises a display tools engine 1350 by which the engine 1300 is to cause display of patient management information on a user interface. As shown in FIG. 12, in some examples the display tools engine 1350 may comprise a summary parameter 1352 to provide a summary of various (selectable) usage parameters. One example implementation of summary parameter 1352 (FIG. 12) comprises the parameters which are displayed within box 1600 in FIG. 13, such as the above-described parameters 1620, 1622, 1630, 1632, 1634, 1636, 1638 of summary portion in FIG. 13. In some examples, the Summary (box 1600) also may display a date range 1610, which specifies and displays a date range for which the values of the parameters were obtained.

As further shown in FIG. 12, the nightly usage per period parameter 1354 (of display tools engine 1350) may cause display of nightly usage information for a selectable period (e.g. 30 days, 60 days, etc.), with one example implementation being provided in the user interface 1500 in FIG. 13 as nightly utilization portion 1700, which includes various indicators 1710, 1720, 1730, and chart 1750. Indicators 1710, 1720 allow selection of and/or display of the selectable period of patient information to be reported, displayed, etc. Meanwhile, indicator 1730 provides an array of indicators 1732A-1732E which may be used to enhance nightly usage information displayed in chart 1750. In some examples, the indicators 1732 may comprise an appointment indicator 1732A, an Epworth Sleepiness Scale (ESS) indicator 1732B, a virtual check-in (VCI) indicator 1732C, a sleep study (SS) indicator 1732D, and/or other type indicator 1732D. Further details regarding such indicators are provided below in context with graph 1750.

As further shown in FIG. 13, in some examples a nightly utilization chart 1750 may comprise a time scale 1752 (e.g. hourly markers on a Y axis) relative to which each night's stimulation therapy usage may be displayed. Chart 1750 also may comprise a selectable time period scale 1754 (e.g. days, weeks, months, and the like on an X axis) for which the nightly usage information is displayed.

Chart 1750 displays an array (or plurality) 1760 of nightly usage indicators 1762, which may take the form of bars, columns, and the like. In some examples, each nightly usage indicator 760 comprises a start portion 1770, therapy portion(s) 1772, and a pause portion 1774 (as applicable). Of course, such indicators and indicator portions may provide more granular information, such as the exact time each therapy portion started, stopped, exact times at which the pause portion started, stopped, etc. As apparent from FIG. 13, via the nightly usage indicators 1762, one can quickly visually see patterns of stimulation therapy usage.

As further shown in FIG. 13, in some examples chart 1750 also may include display of at least some of the indicators 1732A-1732E, which may be used to tag or mark various aspects of the nightly usage, such as when a sleep study (Sleep S, indicator 1732D) occurred, when a virtual check-in (VCI, indicator 1732C) occurred, and the like. In some examples, chart 1750 also may comprise windows 1780 (designated via dashed box), which may identify weekends, during which a patient may sometimes exhibit usage patterns which differ from their usage patterns during weekdays.

With further reference to FIG. 12, the display tools engine 1350 (of patient management engine 1300) may comprise an amplitude changes parameter 1356 by which patient changes in stimulation therapy amplitude may be displayed. One example implementation of the amplitude changes parameters 1356 is provided as amplitude changes portion 1800 of user interface 1500, as shown in FIG. 13. In some examples, the amplitude changes portion 1800 may comprise a graph or chart which provides a plot 1810 (shown as a solid line) of stimulation therapy amplitude over a selectable time period (e.g. 30 days, 60 days, etc.). The chart also may comprise a display of lower and upper limits on an amplitude value selectable by the patient, which are shown via dashed lines as 1822, 1820, respectively.

In some examples, the amplitude changes portion 1800 also may comprise a patient changes/week indicator 1830, which provides a metric of how often the patient is changing stimulation therapy amplitude within a time period (e.g. one week). This indicator may be expressed as an average, mean, or the like. The number of changes per week may sometimes provide an indication to a clinician of whether the patient finds the stimulation therapy to be comfortable, effective, etc.

With further reference to the patient management engine 1300 in FIG. 12, in some examples the display tools engine 1350 may comprise an outcomes parameter 1358. In one example implementation the outcomes parameter 358 (FIG. 12) may be provided in the user interface 2200 of FIG. 14 as an outcomes/effectiveness portion 2210, which provides a summary, details, etc. regarding various performance metrics regarding patient outcomes, therapy effectiveness (i.e. efficacy), etc. regarding the patient's stimulation therapy. As previously noted, in some examples some of the display tools, patient information, etc. appearing in the user interface 2200 in FIG. 14 may be displayed along with at least some of the display tools, patient information, etc. in user interface 1500 in FIG. 13.

With further reference to the user interface 2200 in FIG. 14, in some examples the outcomes/effectiveness portion 2210 may comprise an apnea-hypopnea index (AHI) portion 2220, which displays a patient's AHI relative to various types (2224) of events and/or stages in the patient's progression from being a candidate, upon implantation, and different points in time thereafter. In some examples, these types 2224 may comprise pre-implant, titration, after titration, home sleep test, formal sleep study (polysomnography PSG), and the like. For each type 2224, the AHI portion 2220 may display a date, a treatment AHI, and a total AHI.

With further reference to the user interface 2200 in FIG. 14, in some examples the outcomes/effectiveness portion 2210 may comprise an Epworth Sleepiness Scale (ESS) portion 2240, which displays a patient's ESS relative to various types (2225) of events, stages, etc. In general terms, the ESS score may provide an indication of a patient's daytime sleepiness (i.e. conversely, alertness), which is an indication of the patient's sleep quality, which in turn may be indicative of the efficacy of the stimulation therapy, patient adherence, etc. In some examples, these types 2225 may comprise pre-implant, therapy, and the like. For each event or stage under the heading type 2225, the ESS portion 2240 may display a date, an ESS score. Via such information, a clinician may observe changes in the ESS score depending on various stimulation therapy settings which may operable at different points in time, whether the ESS score is improving over time with continued stimulation therapy, and the like.

With further reference to the patient management engine 1300 in FIG. 12, in some examples the display tools engine 1350 may comprise an equipment parameter 1360. In one example implementation the equipment parameter 1360 (FIG. 12) may be provided in the user interface 2200 of FIG. 14 in a settings portion 2250, which may comprise a system information portion 2260 and generator summary portion 2270. As shown in FIG. 14, the system information portion 2260 may display information regarding a type 2261 of equipment (e.g. stimulation lead, sensor lead, pulse generator), with such information including model numbers, serial numbers, location in body, and the like. Meanwhile, the generator summary portion 2270 may provide more detailed information regarding the pulse generator such as, but not limited to, battery status (e.g. good, bad, high, low, etc.), and the like.

With further reference to the patient management engine 1300 in FIG. 12, in some examples the display tools engine 1350 may comprise a programming function 1370, which may comprise a stimulation parameter 1372, a sensing parameter 1374, and a thresholds parameter 1376. In one example implementation the programming function 1370 (FIG. 12) may be provided in the user interface 2200 of FIG. 14 in a programming portion 2280.

As shown in FIG. 14, and as driven via the stimulation parameter (1372 in FIG. 12), the programming portion 2280 of user interface 2200 may comprise a date range indicator (and selector) 2282 regarding which patient information is displayed. In some examples, a stimulation settings portion 2290 (of programming portion 2280) may comprise a listing of different types 2300 of stimulation settings for which their respective values are listed under an incoming value column 2312 and a final value column 2314. In some examples, the types 2300 may comprise items 2310 such as amplitude (Volts), patient control (V), pulse width (microseconds), rate (Hertz), Start Delay (minutes), and the like. At 2311, the stimulation settings portion 2290 also may display which electrode(s), electrode configuration, is being used.

In some examples, for at least some of the listed items 2310, a changes made indicator 2313 may be displayed, which may act as a flag intended to catch the attention of the clinician regarding stimulation settings which have changed, per patient use (or for other reasons).

In some examples, the stimulation settings portion 2290 may comprise an advanced indicator 2316 which a clinician may activate to observe advanced stimulation settings within user interface 2200 in FIG. 14.

As further shown in FIG. 14, and as driven via the sensing parameter 1374 (of display tools engine 1350) in FIG. 12, the sensing portion 2350 (of programming portion 2280 of user interface 2200) may comprise a listing of different types 2360 of sensing settings for which their respective values are listed under an incoming value column 2312 and a final value column 2314. In some examples, the types 2360 may comprise items such exhalation, off period (e.g. as a percentage), maximum stimulation time (e.g. seconds), whether or not the sensing signal has become inverted, and the like.

As further shown in FIG. 14, and as driven via the thresholds parameter (1376 in FIG. 12), the stimulation thresholds portion 2380 of user interface 2200 may comprise a current settings portion 2382 which may comprise a listing of value of the current stimulation settings, such as (but not limited to) the parameters of rate (Hz), Pulse Width (microseconds), maximum stimulation pulse (e.g. seconds), as well as an identification of a current electrode(s) configuration 2383).

In some examples, the stimulation thresholds portion 2380 may comprise a listing of different types 2385 of stimulation threshold settings for which their respective values are listed under an amplitude column and an airway status column (e.g. opening). In some examples, the types 2385 may comprise items such amplitude and patient controllable amplitude.

In a manner similar to the stimulation settings portion 2290, the stimulation thresholds portion 2380 of user interface 2200 in FIG. 14 may comprise a changes made indicator 2313 to indicate that changes were made regarding the stimulation amplitude. It will be further understood that the changes made indicator 2313 may be displayed in association with other parameters, functions, etc. of user interface 2200 in FIG. 14 (and/or of user interface 1500 in FIG. 13).

With reference again to FIG. 12, the display tools engine 1350 may comprise an other parameter 1377 by which other parameters relating to stimulation therapy, patient management, etc. may be automatically or selectively displayed on a user interface. For instance, in some examples, via the other parameter 1377 of display tools engine 1350, upon activation of any one or more of the indicators (e.g. 1532, 1534, etc.) of array 1530 of user interface 1500 in FIG. 13, the subject matter associated with a respective indicator may be displayed on user interface 1500 (FIG. 13), 2200 (FIG. 14), and/or combination thereof. This arrangement enables customization of the respective user interfaces 1500 (FIG. 13), 2200 (FIG. 14) according to the preferences of the clinician, needs of the particular patient, stage of care of the particular patient, and the like. At least some aspects of example implementations of the other parameter 1377 of the display tools engine 1350 (FIG. 12) will be later described further in association with at least FIGS. 15A-15B with regard to at least the virtual check-in (Virtual C I) indicator 1536. As a further example, activation of the ESS indicator 1534 may result in display of patient information regarding an Epworth Sleepiness Scale (ESS), such as the ESS portion 2240 in user interface 2200, as shown in FIG. 14.

Accordingly, the above-described various features of the user interfaces 1500 (FIG. 13), 2200 (FIG. 14), as driven via the various functions and parameters of the display tools engine 1350 in FIG. 12, provide a rich environment by which a clinician may manage patient care. For instance, in some examples, upon a clinician viewing the various displayed patient information in user interface 1500 in FIG. 13 regarding nightly usage and/or amplitude changes and in user interface 2200 in FIG. 14 regarding outcomes/effectiveness and settings (e.g. stimulation, sensing, and the like), the clinician can make an assessment regarding patient symptoms, patient adherence, and/or therapy efficacy (e.g. patient outcomes). Based on the assessment, the clinician may order further patient education, changes to stimulation therapy programming, and the like, as desired. At least some further details regarding this information-rich environment for patient management are described in association with at least FIGS. 15A-15B, among other examples throughout the present disclosure.

As further shown in FIG. 12, the communication engine 1380 (of patient management engine 1300) may control, affect, facilitate, etc., at least in part, communications among various elements of the example arrangement 1100 in FIG. 11, such as clinician portals 1155 (of clinician devices 1150), patient apps 1112 (of devices 1110), patient remote 1120, etc.

In some examples, the communication engine 1380 may comprise a link function 1382 to specify and/or drive communications per a selectable link specified via a type parameter 1384 and/or a status parameter 1386. The type parameter 1384 may comprise various types of communication links, which may be wired or wireless (e.g. internet, Bluetooth, peer-to-peer, LAN, WAN, and the like).

In some examples, the communication engine 1380 in FIG. 12 also may comprise a tools function 1390 providing various communication tools including, but not limited to, a share parameter 1392 by which various patient management information, actions, etc. may be shared, such as among members of a care team (e.g. the different clinicians associated with the different portals 1160 in FIG. 11). Similarly, via parameter 1392, such information also may be shared with a patient, insurer, other.

It will be understood that the information displayed, accessed, input, etc. on the user interface 1500 (FIG. 13) and 2200 (FIG. 14) may be presented in a wide variety of formats, with the examples shown in FIGS. 13 and 14 being just one example format.

FIG. 15A is a block diagram schematically representing an example patient app 2512, which may comprise at least some of substantially the same features and attributes as the patient app 1112 (on a patient device 1110) in FIG. 11, except further comprising additional parameters, functions, etc. such as a sleepiness scoring tool 2514 and/or other tools 2526. In some examples, via the patient app 2512 a patient may enter information regarding their experience (e.g. fatigue, cognitive acuity, etc.) according to a sleepiness scale such as, but not limited to, the Epworth Sleepiness Scale (ESS).

One example implementation of displaying the results of a patient's subjective experience as conveyed via the sleepiness scoring tool 2524 of patient app 2512 (e.g. on a patient device 1110 in FIG. 11) may comprise displaying the Epworth sleepiness scale portion 2240 in the outcomes/effectiveness portion 2210 of user interface 2200 of FIG. 14. In some examples, the display (or lack thereof) of the ESS portion 2240 may be controlled (e.g. selected by the clinician) via the ESS indicator 1534 in general portion 102 of user interface 1500 in FIG. 13. When the ESS portion 2240 is displayed, it may become juxtaposed with objectively obtained information such as AHI data in the apnea-hypopnea index (AHI) portion 2220, as shown in FIG. 14 However, in some examples, upon its display in the user interface (e.g. 1500 in FIG. 13, 2200 in FIG. 14), the ESS portion 2240 may be juxtaposed (or generally co-located) with elements such as the nightly utilization portion 1700 and/or amplitude changes portion 1800 in FIG. 13. Via such arrangements, a clinician may experience a convenient integration of both the objective information regarding patient usage of stimulation therapy (e.g. via portions 1700, 1800 of FIG. 14) and with the subjective information provided via the ESS portion 2240 or other subjective information, such as provided via the virtual check-in tool 2520 (FIG. 15B).

In some examples, the other tools function 1526 of the patient app 2512 in FIG. 15A may comprise just one or a wide range of patient education tools such as (but not limited to) a success story library, general sleep apnea/therapy information, and the like. In some examples, the other tools function 2526 also may comprises tools by which a patient may convey their subjective experiences to a clinician, and which then can be displayed with other information (e.g. objective, other) on user interfaces 1500 (FIG. 3), 2200 (FIG. 4).

FIG. 15B comprises a block diagram schematically representing an example patient survey engine 2540. In some examples, the example patient survey engine 2540 may comprise a portion of, and/or be implemented via, control portion 2600 in FIG. 17A. In general terms, the patient survey engine 2540 controls and supports operation of a patient survey (e.g. function 2522 in FIG. 15A) displayable on a user interface (e.g. 2640 in FIG. 17B), such as via an app 2512 in FIG. 15A (or app 1112 in FIG. 11) of a display screen of a computing device (e.g. 1110 in FIG. 11), which may be mobile such as a mobile phone, tablet, etc. However, it will be understood an example patient survey (e.g. per function 2522) may be displayed and provided on a user interface of a stationary computing device in some examples. In some examples, among display of other information associated with the virtual check-in function 2520 (FIG. 15A), the results of the patient survey may be displayed on the user interface 1500 (FIG. 13), 2200 (FIG. 14) upon activation of the Virtual check-in (Virtual C I) indicator 1536 in FIG. 13.

As shown in FIG. 15B, in some examples the patient survey engine 2540 may comprise parameters to query a patient regarding: (A) consistent usage (2541) (“are you using the device every night?); (B) comfort (2542) (“is the stimulation comfortable?”); (C) hindrances (2544); (D) device visible indicators (2550); (E) snoring (2554) (“has your snoring improved?”); (F) feeling (2556) (e.g. “do you feel tired?”), and/or other parameters (2558).

In some examples, the hindrances parameter 2544 may relate to (and track) factors which are preventing (or perceived to be preventing) a patient from employing therapy every night. At least some example hindrance factors may comprise: (1) the stimulation therapy is uncomfortable; (2) it wakes me up; (3) I forget to use it every night, and/or (4) other reasons. In some examples, the device visible indicators parameter 2550 may relate to (and track) a patient query of what the patient observes regarding visible indicators on a therapy component, such as a patient remote control (e.g. 1120 in FIG. 11). The visible indicators may provide an indication of the amplitude strength being used, in some examples. In some examples, other types of therapy or usage parameters which may be conveyed via visible indicators on a housing of the patient remote control or other therapy peripheral.

It will be understood that such example patient surveys are not limited to the exact words noted above and may use different expression, terms, etc. while still seeking and obtaining the same type of information. Moreover, as evident by the other parameter 2558, the example patient survey provided via patient survey engine 2540 in FIG. 15B is not strictly limited to the above-noted example queries.

As previously noted, the results of the patient survey (e.g. taken via patient device 1110) may be communicated from the patient app (e.g. 1112 in FIG. 11; 1512 in FIG. 15A) to one of the clinician devices 1150 (FIG. 11) via resource 1130 for display on user interface 1500 (FIG. 13), 2200 (FIG. 14) in association with activation of (or an already activated) virtual check-in (Virtual C I) indicator 1536 in user interface 1500 (FIG. 13).

In some examples, displaying the results of a patient's subjective experience as conveyed via the patient survey 2522 (FIG. 15A) of patient app 2512 (e.g. on a patient device 1110 in FIG. 11) may be provided in the outcomes/effectiveness portion 2210 of user interface 2200 in FIG. 14. When the results of the patient survey (from the virtual check-in) are displayed, the results may be juxtaposed with objectively obtained information such as AHI information in AHI portion 2220 in user interface 2200 of FIG. 14 However, in some examples, a display of the results of the patient survey (obtained via an virtual check-in) may be juxtaposed (or generally co-located) with elements such as the nightly utilization portion 1700 and/or amplitude changes portion 1800 in FIG. 13. Via such arrangements, a clinician may experience a convenient integration of both objective information regarding patient usage of stimulation therapy (e.g. via portions 1700, 1800 of FIG. 14) and subjective information provided via the results of patient survey 2522 or other subjective information. For instance, by seeing both the objective information (e.g. nightly usage patterns (chart 1750 in FIG. 13), quantitative metrics re how often nightly usage occurs (1620 in FIG. 13), for how long each night (e.g. usage per night greater than 4 hours (1622 in FIG. 13), etc.) and the subjective information (e.g. survey questions, such as “is the stimulation comfortable?”, “do you feel tired?”, etc.) on a single user interface (or related user interfaces), a clinician becomes positioned to make more sophisticated observations regarding patient symptoms, patient adherence, therapy efficacy, and the like. Such observations may then be used to confirm or modify stimulation therapy settings or other parameters as part of treating the patient. In some examples, such observations might otherwise not occur (or might occur much slower) if the objective patient usage information, objective outcomes, etc. were not displayed together with the above-noted subjective patient information in the manner provided in at least some examples of the present disclosure.

FIG. 16 is a flow diagram schematically representing an example method 2580 of patient management. In some examples, method 2580 may be implemented via at least some the example arrangements, patient remote, patient app, processing resource, clinician portals, engines, parameters, functions, user interfaces, control portions, etc., as described in association with at least FIGS. 11-15 and 17A-17B. In some examples, method 2580 may be implemented via at least some elements, arrangements, patient remote, patient app, processing resource, clinician portals, engines, parameters, functions, user interfaces, control portions, etc. other than those described in association with at least FIGS. 11-15 and 17A-17B.

As shown at 2582 in FIG. 16, in some examples method 2582 comprises receiving, via a processing resource, patient information from a patient app. In some such examples, the method may comprise the patient app receiving the patient information from a patient remote control. The patient remote control may receive at least some of the patient information from an implantable medical device and/or may produce some of the patient information from the patient remote control tracking and storing patient usage of the patient remote control.

As shown at 2584 in FIG. 16, in some examples method 2580 comprises displaying, via the processing resource, the patient information on a clinician portal. The clinician portal may comprise various display tools for displaying the patient information in a manner conducive to enhancing patient adherence, efficacious therapy, and/or improving patient outcomes.

FIG. 17A is a block diagram schematically representing an example control portion 2600. In some examples, control portion 2600 provides one example implementation of a control portion forming a part of, implementing, and/or generally managing the example arrangements, the implantable medical devices (IMDs, e.g. IPG), cloud resources, patient remotes, patient apps, programmers, user interfaces, control portion, instructions, workflows, engines, functions, parameters, and/or methods, as described throughout examples of the present disclosure in association with FIGS. 11-16. In some examples, control portion 2600 includes a controller 2602 and a memory 2610. In general terms, controller 2602 of control portion 2600 comprises at least one processor 2604 and associated memories. The controller 2602 is electrically couplable to, and in communication with, memory 2610 to generate control signals to direct operation of at least some of the example arrangements, IMDs, cloud resources, patient remotes, patient apps, programmers, user interfaces, control portion, instructions, workflows, engines, functions, parameters, and/or methods, as described throughout examples of the present disclosure. In some examples, these generated control signals include, but are not limited to, employing instructions 2611 and/or information 2612 stored in memory 2610 to at least direct and manage sleep disordered breathing (SDB) care (e.g. sensing, stimulation, related patient management, etc.) in the manner described in at least some examples of the present disclosure. In some instances, the controller 2602 or control portion 2600 may sometimes be referred to as being programmed to perform the above-identified actions, functions, etc.

In response to or based upon commands received via a user interface (e.g. user interface 2640 in FIG. 17B) and/or via machine readable instructions, controller 2602 generates control signals as described above in accordance with at least some of the examples of the present disclosure. In some examples, controller 2602 is embodied in a general purpose computing device while in some examples, controller 2602 is incorporated into or associated with at least some of the example arrangements, IMDs, cloud resources, patient remotes, patient apps, programmers, user interface, control portion, instructions, workflows, engines, functions, parameters, and/or methods, etc. as described throughout examples of the present disclosure.

For purposes of this application, in reference to the controller 2602, the term “processor” shall mean a presently developed or future developed processor (or processing resources) that executes machine readable instructions contained in a memory or that includes circuitry to perform computations. In some examples, execution of the machine readable instructions, such as those provided via memory 2610 of control portion 2600 cause the processor to perform the above-identified actions, such as operating controller 2602 to implement patient management and care via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 2610. The machine readable instructions may include a sequence of instructions, a processor-executable machine learning model, or the like. In some examples, memory 2610 comprises a computer readable tangible medium providing non-volatile storage of the machine readable instructions executable by a process of controller 2602. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 2602 may be embodied as part of at least one application-specific integrated circuit (ASIC), at least one field-programmable gate array (FPGA), and/or the like. In at least some examples, the controller 2602 is not limited to any specific combination of hardware circuitry and machine readable instructions, nor limited to any particular source for the machine readable instructions executed by the controller 2602.

In some examples, control portion 2600 may be entirely implemented within or by a stand-alone device.

In some examples, the control portion 2600 may be partially implemented in one of the example arrangements, IMDs, patient remotes, patient apps, programmers, etc. and partially implemented in a computing resource separate from, and independent of, the example arrangements, IMDs, cloud resources, patient remotes, patient apps, programmers, etc. but in communication with such example arrangements, etc. For instance, in some examples control portion 2600 may be implemented via a server accessible via the cloud and/or other network pathways. In some examples, the control portion 2600 may be distributed or apportioned among multiple devices or resources such as among a server, an example arrangement, and/or a user interface.

In some examples, control portion 2600 includes, and/or is in communication with, a user interface 2640 as shown in FIG. 17B. In some examples, user interface 2640 comprises a user interface or other display that provides for the simultaneous display, activation, and/or operation of at least some of the example arrangements, IMDs, cloud resources, patient remotes, patient apps, programmers, user interface, control portion, workflows, instructions, engines, functions, parameters, and/or methods, etc., as described in association with FIGS. 1-6. For instance, the user interface 1500 (FIG. 13) and/or user interface 2200 (FIG. 14) may comprise example implementations of user interface 2640 in FIG. 17B. In some examples, at least some portions or aspects of the user interface 2640 (e.g. 1500 in FIG. 13, 2200 in FIG. 14) are provided via a graphical user interface (GUI), and may comprise a display 2642 and input 2644.

FIG. 18 is a block diagram schematically representing an example arrangement 3100 of a method (and/or device) for patient care in treating sleep disordered breathing (SDB), which includes aspects of patient management, stimulation therapy, and third party diagnostic/monitoring. In some examples, the example arrangement 3100 may comprise at least some of substantially the same features and attributes as the example arrangement of FIGS. 1-10 and/or FIGS. 11-17B.

As shown in FIG. 18, the example arrangement 3100 may comprise a third party device 3110 and/or second resource 3130B in addition to the previously described patient device 1110 (with patient app 1112), patient remote 1120, implantable medical device 1125, clinician device 1150 (with portal 1155), and first resource 3130A (e.g. 1130 in FIG. 11).

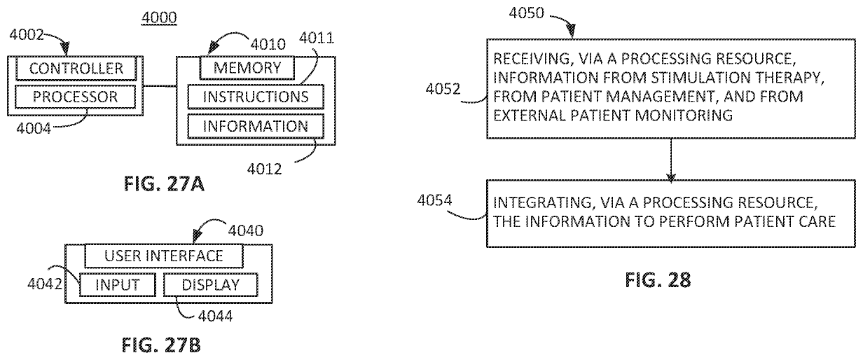

In some examples, the first resource 3130A may comprise at least some of substantially the same features and attributes as the resource 1130 as previously described in association with at least FIG. 11. Accordingly, among other aspects, in some examples, first resource 3130A may comprise at least a portion or, and/or an example implementation of, the control portion 2600, 4000 (FIGS. 17A, 27A, respectively) and user interface 2640, 4040 (FIGS. 17B, 27B, respectively). In some examples, an entity providing first resource 3130A may comprise a device (e.g. implantable medical device) manufacturer, device supplier, or entity contracted by the device manufacturer. The first resource 3130A may be hosted by a server, processing resource, etc. available via the cloud computing network 3120 (e.g. the internet, World Wide Web, and/or other network communication link).

In some examples, the third party device 3110 may comprise a device which is external to a patient. In some such examples, the third party device 3110 may be selectively worn by the patient such as around the wrist or on a finger, an arm, an ankle, a torso, a head, a neck, etc. of the patient. In some of these examples, third party device 3110 may take the form of a wrist-watch, such as a smart watch. However, in some such examples, the externally-located third-party device 3110 may not be worn by the patient, but be positioned in a proximity to the patient sufficient for sensing to enable monitoring and/or diagnosis. In some of these examples, the third party device 3110 may comprise a patient support such as, but not limited to, a mattress, bed, chair, and the like.

In some examples, the third party device 3110 may comprise some components worn by the patient and some components which placed in close proximity to, but not worn, by the patient.

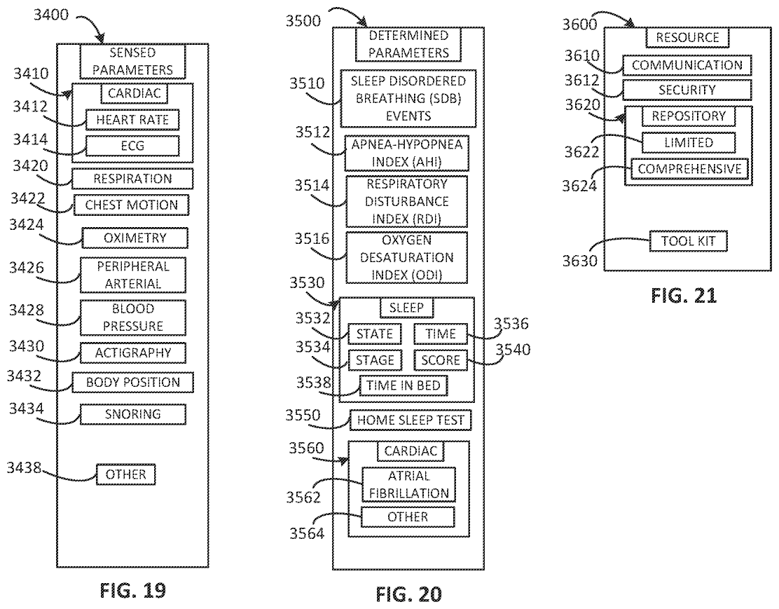

Whether worn on the patient and/or positioned near the patient, sensing components of the third party device 3110 become oriented in sensing relation to one or more portions of the patient's body so as to facilitate sensing various physiologic phenomenon such as (but not limited to) the sensing parameters described in association with at least FIG. 19.

As further shown in FIG. 18, in some examples the third party device 3110 may communicate directly with patient device 1110 (hosting a patient mobile app 1112) such that the parameters (e.g. information) sensed (FIG. 19) and/or parameters determined (FIG. 20) by the third party device 3110 may be integrated directly into, and/or be complementarily combined with, SDB care-related information on the patient device 1110, such as on patient mobile app 1112. This SDB care-related information may comprise patient management information and/or stimulation therapy-related information, as further described below, or other information.

In some examples, communication (as represented by arrow 3117) between the third party device 3110 and the patient mobile app 1112 (hosted on patient device 111) may be performed via a wireless communication protocol (e.g. Bluetooth, infrared, near-field communication) and/or via a wired connection between the respective devices 3110 and 1110. In some instances, this communication pathway may sometimes be referred to as a direct communication pathway.

In some examples, the third party device 3110 may comprise a general consumer product which is not specifically dedicated for use with patient device 1110, patient remote 1120, and/or implantable medical device 1125.

As further shown in FIG. 18, in some examples the example arrangement 3100 may comprise a second resource 3130B, which provides operational support for diagnostic (3112), monitoring (3114) functions of the third party device 3110 and/or for communication with other devices such as patient mobile app 1112 (on device 1110). In some such examples, the second resource 3130B may provide an operational framework by which information sensed, determined, and/or stored by the third party device 3110 may be communicated between the third party device 3110 and the second resource 3130B. Accordingly, in some of these examples, the second resource 3130B may comprise an operations engine hosted by a server, processing resource, etc. available via the cloud computing network 3120.

Via this arrangement, the information sensed and/or determined by the third party device 3110 may be communicated to the mobile patient app 1112 by communication implemented via the cloud 3120 (or other network communication link) between the second resource 3130B and the first resource 3130A. In some examples, the second resource 3130B may sometimes be referred to as a back-end of the diagnostic/monitoring functions (on third party device 3110) and the first resource 3130A sometimes being referred to as a back-end of the patient mobile app 1112 (hosted by patient device 1110) such that communication between the first and second resources 3130A, 3130B may sometimes be referred to as back end communication between the third party diagnostic/monitoring functions and the patient mobile app.

Among other aspects, this back end communication pathway may provide one pathway by which the third party diagnostic/monitoring information may be integrated with patient management information and/or stimulation therapy (e.g. information and stimulation).

In some examples, the example arrangement 3100 may comprise a third resource 3130C which may act an intermediary to facilitate or coordinate communication between the third party device 3110 and the patient mobile app 1112 (on patient device 1110) either directly such as via pathway 3117 or via a back end pathway between the second resource 3130B and the first resource 3130A. In some such examples, the third resource 3130C may be provided by a communication service platform provider such as, but not limited to, an entity like Apple, Inc. providing the Apple® Healthkit®. At least some aspects regarding such a resource are further described in association with at least FIG. 21.

With these various aspects of the example arrangement 3100 of FIG. 18 in mind, in some examples the first resource 3130A may provide a framework which at least partially coordinates the integration of stimulation therapy (and related information), patient management, and externally monitored (e.g. sensed or received) information such as from third party device 3110. In some such examples, the framework may comprise at least some of substantially the same features and attributes as, and/or an example implementation of, control portion 4000 in FIG. 27A (and user interface 4040 in FIG. 27B). In some of these examples, the control portion 4000 (FIG. 27A) and/or user interface 4040 (FIG. 27B) may incorporate and/or comprise at least some of substantially the same features and attributes as (and/or an example implementation of) the previously-described control portion(s) and user interface(s) in association with at least FIGS. 17A-17B (e.g. 2600, 2640) and/or FIGS. 9-10 (e.g. 1050, 1070).

Among other aspects, in some examples the example arrangement 3100 in FIG. 18 (and related example implementations in FIGS. 19-28) may overcome challenge in non-example arrangements in which patient management systems (e.g. involving nightly usage data, patient outcomes, scheduling, etc.) may operate separately from stimulation therapy devices/systems and/or separately from externally monitored patient information (for diagnostics or monitoring) provide by a third party device. In such non-example arrangements, these different forms of activity (e.g. on-going therapy), clinical information, and monitored patient parameters (e.g. sensed data) generally remain disparate from each other due to the bulk of the information, different forms in which the information is gathered or produced, lack of developed communication and care pathways, etc.

In contrast, the example arrangement 3100 in FIG. 18 (and related example implementations in FIGS. 19-28) establishes a patient care therapy and communication framework (which may be complementary with or inclusive of the various examples of the present disclosure in association with at least FIGS. 1-10 and/or FIGS. 11-17B) to integrate previously disparate aspects of patient management, stimulation therapy, and externally monitored patient information.

FIG. 19 is a block diagram schematically representing example sensing parameters associated with at least a third party diagnostic/monitoring portion. In some examples, the example arrangement 3100 may comprise at least some of substantially the same features and attributes as the example arrangement of FIGS. 1-10 and/or FIGS. 11-17B.

In some examples, the sensing parameters may be sensed by, and/or received at, the third party device 3110.

In general terms, these sensing parameters are in addition to any parameters sensed by the implantable medical device 1125, patient remote 1120, and/or patient mobile app 1112 (hostable on device 1110), which are generally considered part of, or related to, a therapy system owned or provided by an entity other than the third party which sells, manages, supports, etc. the third party device 3110. However, in some examples, via contractual relationship or other mechanism, the third party device 3110 may be controlled by the same entity which controls, manufactures, etc. the implantable medical device 1125, patient remote 1120, and/or patient mobile app 1112 (hostable on device 1110).

In some examples, the sensing parameters may comprise cardiac parameters 3410 such as, but not limited to, a heart rate (HR) 3412, an electrocardiogram (ECG) 3414, and/or other cardiovascular phenomenon.

In some examples, the sensing parameters 3400 may comprise parameters of respiration 3420, chest motion 3422, and/or oximetry 3424. In some examples, the sensed chest motion 3422 may comprise just one of several modalities to sense respiration 3420. The oximetry parameter 3424 may be sensed via pulse oximetry, such as a finger-mountable pulse oximetry unit or other modality.

In some examples, the sensing parameters 3400 may comprise parameters of a peripheral arterial signal 3426, blood pressure 3428, and/or actigraphy 3430.

In some examples, the sensing parameters 3400 may comprise parameters of body position 3432, snoring 3434 (or other acoustic parameters), and/or other 3438 parameters of physiologic phenomenon or environmental phenomenon, which may relate to patient care for treating sleep disordered breathing (SDB).

FIG. 20 is a block diagram schematically representing example determined parameters 3500 associated with at least a third party diagnostic/monitoring device 3110 and related support/services.

In general terms, the determined parameters 3500 may be determined from, or in association with, at least some of the sensed parameters 3400 of FIG. 19.

In some examples, the determined parameters 3500 may comprise sleep disordered breathing (SDB) events 3510, which may comprise apnea events, hypopnea events, and or other detectable events. The apnea events may comprise events of obstructive sleep apnea, central sleep apnea, and/or mixed sleep apnea.

In some examples, the determined parameters 3500 may comprise various indices for quantifying an intensity or severity of such SDB events such as, but not limited to, an apnea-hypopnea index (AHI) 3512, respiratory disturbance index (RDI) 3514, and/or an oxygen desaturation index (ODI) 3516.

In some examples, the determined parameters 3500 may comprise various parameter for quantifying aspects of sleep quality such as, but not limited to, sleep state 3532, sleep stage 3524, time 3536, score 3540, and time-in-bed 3538. In some such examples, the sleep state parameter 3532 may comprise a sleep-wake status, sleep start time, sleep stop time, sleep pause times, and the like. The time parameter 3536 may relate to total sleep time, total stimulation therapy time, and/or other time parameters related to sleep. In some examples, the score parameter 3540 may relate to a sleep score which provides information about sleep quality. In some examples, the time-in-bed parameter 3538 may provide a total time-in-bed within an intended sleep period (e.g. 10 pm to 6 am), within a twenty-four hour period, etc. Among other aspects, the time-in-bed parameter 3528 may help provide context for the start times, stop times, number and duration of pause times, and/or total therapy-on duration, which in turn may be used to evaluate therapy efficacy, patient adherence, and related care parameters.

In some examples, the determined parameters 3500 may comprise a home sleep test (HST) parameter 3550, which may comprise information obtained from a sleep study performed in a home study using at least some of the sensed parameters 3400 in FIG. 19 obtainable via third party device 3110. In some such examples, the home sleep study may approximate a formal polysomnongraphy (PSG) study.

In some examples, the determined parameters 3500 may comprise cardiac parameters 3560 such as, but not limited to, atrial fibrillation 3562 and/or other parameters 3562, which may relate to other arrhythmias or other indicators of negative and/or positive cardiac health, which may be used to evaluate, adjust, etc. aspects of sleep disordered breathing (SDB) care.

FIG. 21 is a block diagram schematically representing an example resource 3600. In some examples, the example resource 3600 may comprise at least some of substantially the same features and attributes as, and/or an example implementation of, the example resources (e.g. 3130A, 3130B, 3130C) of FIG. 18, of FIG. 11 (e.g. 1130), and/or FIG. 1.

As shown in FIG. 21, in some examples the resource 3600 may comprise a communication framework 3610 to facilitate communication via network communications (e.g. Web, cloud, and the like), hosting on a server, etc. In some examples, the resource 3600 may comprise a security component 3612 to generally safeguard customer or patient data, comply with HIPAA regulations, and/or other regulatory frameworks.

In some examples, the resource 3600 may comprise a repository 3620 of a patient's data, which may be limited via parameter 3622 to just data sensed (FIG. 19) and/or determined (FIG. 20) by devices (e.g. 3110) and/or services proprietary to a third party diagnostic/monitoring provider. However, in some examples, the repository 3620 may receive, transmit, store, and/or manage other data which may be more comprehensive via parameter 3624, such as data from parameters sensed and/or determined via devices and/or services other than the third party diagnostic/monitoring devices (and/or services) like device 3110 in FIG. 18. In some such examples, this other data may be sensed and/or determined via patient remote 1120, the implantable medical device 1125, and/or sensors in communication with the implantable medical device 1120, the patient remote 1120, and/or patient mobile app 1112 (hosted by device 1110).

As further shown in FIG. 21, in some examples the resource 3600 may comprise a developer tool kit 3630 by which at least one of the first, second, and third resources (3130A, 3130B, 3130C in FIG. 18) may be customized to facilitate communication between, and integration of data between, the third party device 3110, patient mobile app 1112 (hosted on patient device 1110), patient management 1160 (on clinician portal 1155), patient remote 1120, and/or implantable medical device 1125 (via patient remote 1120 and/or patient mobile app 1112.

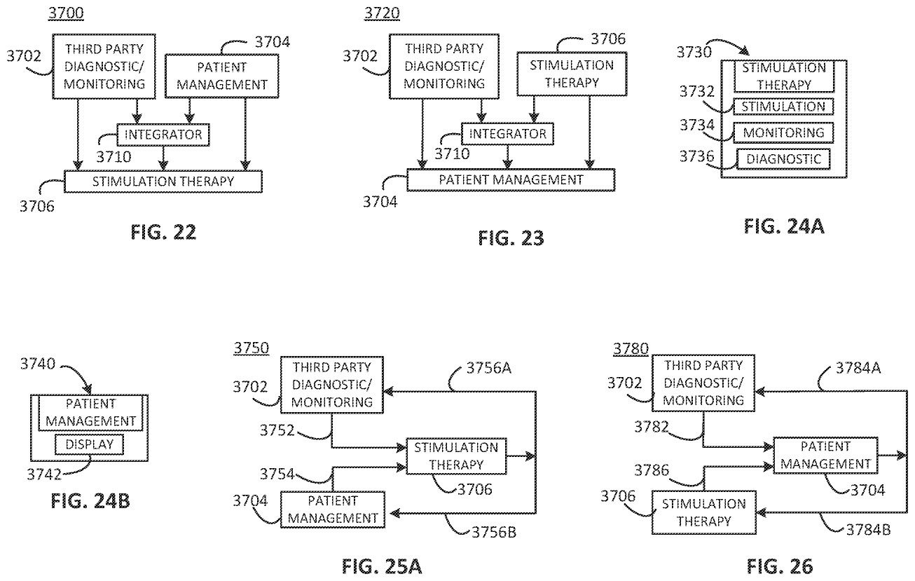

FIG. 22 is a block diagram schematically representing an example method 3700 (and/or example device) for integrating a patient management input and a third-party diagnostic/monitoring input into stimulation therapy implementation. In some examples, the third party diagnostic/monitoring input 3702 (e.g. portion 3702) may be implemented via the third party diagnostic/monitoring device 3110 and/or second resource 3130B in FIG. 18. Meanwhile, in some examples, the patient management input 3704 (e.g. portion 3704) may be implemented via the patient management portion 1160 of clinician portal 1155, patient mobile app 1112, patient remote 1120, and/or first resource 3130A in FIG. 18. In some examples, the stimulation therapy portion 3706 may be implemented via at least the implantable medical device 1125 and/or patient remote 1120.

As further shown in FIG. 22, the third party diagnostic/monitoring input 3702 and the patient management input 3704 may provide input pathways 3703A, 3703B, respectively, to the stimulation therapy portion 3706 to enhance implementing the stimulation therapy. In some examples, both of the third party diagnostic/monitoring input 3702 and the patient management portion input 3704 may provide inputs which are integrated in a complementary manner via an integrator 3710 prior to being provided to the stimulation therapy. Such integration may enhance reconciliation of different classes of input information, any duplicative sensed parameters or determined parameters, etc. which come from the disparate sources of input information.

In some examples, the integrator 3710 may be incorporated within (or as part of) the stimulation therapy implementation 3706 and/or may be a standalone component configured to perform such data integration. In some such examples, the integrator 3710 may be at least partially implemented via at least one of the first, second, or third resources 3130A, 31306, 3130C (FIG. 18) and/or via one of the devices of example arrangement 3100 in FIG. 18 which are in communication with at least one of those respective resources.

FIG. 23 is a block diagram schematically representing an example method (and/or example device) for integrating a third-party diagnostic/monitoring input and a stimulation therapy input into patient management. In some examples, the third party diagnostic/monitoring input 3702 (e.g. portion 3702) may be implemented via the third party diagnostic/monitoring device 3110 and/or second resource 3130B in FIG. 18. Meanwhile, in some examples, the stimulation therapy portion 3706 (e.g. portion 3706) may be implemented may be implemented via at least the implantable medical device 1125 and/or patient remote 1120. In some examples, patient management implementation 3704 may be implemented the patient management portion 1160 of clinician portal 1155, patient mobile app 1112, patient remote 1120, and/or first resource 3130A in FIG. 18.

As further shown in FIG. 23, the third party diagnostic/monitoring input 3702 and the stimulation therapy input 3706 may provide input pathways 3723A, 3723B, respectively, to the patient management implementation 3704 to enhance implementing patient management. In some examples, both of the third party diagnostic/monitoring input 3702 and the stimulation therapy input 3706 may provide inputs which are integrated in a complementary manner via an integrator 3729 prior to being provided to the patient management implementation 3704. Such integration may enhance reconciliation of different classes of input information, any duplicative sensed parameters or determined parameters, etc. which come from the disparate sources of input information.

In some examples, the integrator 3729 may be incorporated within the patient management implementation 3704 and/or may be a standalone component configured to perform such data integration. In some such examples, the integrator 3729 may be at least partially implemented via one of (or a combination of) the first, second, or third resources 3130A, 3130B, 3130C (FIG. 18) and/or via one of the devices of example arrangement 3100 in FIG. 18 which are in communication with those respective resources.

FIG. 24A is a block diagram schematically representing an example stimulation therapy portion. The stimulation therapy portion 3730 comprises at least some of substantially the same features and attributes as, and/or an example implementation of, the stimulation therapy portions in the respective example arrangements of at least FIGS. 22-23, 25-26. As shown in FIG. 24, the stimulation therapy portion 3730 comprises stimulation portion 3732 (e.g. circuitry, firmware, programming, etc.) to deliver an electrical stimulation signal via a stimulation element (e.g. electrode(s)) to airway patency-related tissue (e.g. upper airway patency-related tissue) such as, but not limited to, the hypoglossal nerve, ansa cervicalis-related nerve, and/or other nerve. In some examples, the stimulation therapy portion 3730 may comprise a monitoring portion 3734 and/or diagnostic portion 3736. In some examples, the monitoring portion 3734 may comprise sensing modalities for a wide variety of physiologic phenomenon such as, but not limited to, sensing respiration. In some such examples, the sensed respiration may be used to synchronize the stimulation signal with portions of a sensed respiratory waveform, and therefore provide one type of closed loop stimulation. In some examples, the sensing modalities may be used to evaluate an efficacy of the stimulation therapy. In some examples, the sensing modalities may be omitted for purposes of timing stimulation of targeted tissue, such that the stimulation may sometimes be referred to as open loop stimulation which does not attempt to cause the stimulation to coincide with (e.g. synchronize) with, or be triggered by, certain phases of respiration like inspiration and/or expiration.

The monitoring portion 3734 also may be used to sense other physiologic information such as any of the sensing parameters and/or determined parameters identified (for third party device 3110) in FIGS. 19 and 20 or other parameters which may be sensed or determined which may enhance patient care in treating sleep disordered breathing.

As further shown in FIG. 24A, in some examples the stimulation therapy portion 3730 may comprise diagnostic portion 3736 which develops, tracks, etc. diagnostic information such as (but not limited to) at least some of the determined parameters identified (for third party device 3110) in FIG. 20 for diagnosing diseases, physiologic conditions, etc. related to, and/or in addition to, sleep disordered breathing. For example, such diagnostic information may comprise cardiac information such as (but not limited to) the cardiac information (e.g. 3560) previously described in association with the determined parameters 3500 in FIG. 20. In some such examples, this diagnostic functions of the stimulation therapy portion 3730 may sometimes be referred to as being embedded with (or as part of) the stimulation therapy portion 3730 or as being on-board the stimulation therapy portion 3730.

FIG. 24B is a block diagram schematically representing an example patient management portion 3740 comprising at least some of substantially the same features and attributes as, and/or an example implementation of, the patient management input 3704 (e.g. portion 3704) as described in association with at least FIGS. 22, 23, 25, and/or 26. As shown in FIG. 24B, in some examples the patient management portion 3740 may comprise a display 3742 such as, but not limited to, a user interface like user interface 4040 in FIG. 27B, which may comprise a graphical user interface.

In some examples, the third party diagnostic/monitoring input 3702 (e.g. portion 3702) of FIGS. 22, 23, 25, 26 may comprise a display 3742 in addition to (or instead of) the display 3742 of the patient management portion 3704.

FIG. 25 is a block diagram schematically representing an example method 3750 (and/or example device) for stimulation therapy including closed-loop feedback from a patient management input and a third-party diagnostic/monitoring input. In some examples, the method 3750 (and/or device) may comprise at least some of substantially the same features and attributes as, and/or an example implementation of, the example arrangement 3700 as described in association with at least FIG. 22. As shown in FIG. 25, the example method 3750 may comprise providing a stimulation therapy (via portion 3706) to treat airway patency-related tissue (e.g. upper airway) with results (e.g. efficacy, parameters, etc.) from the therapy being communicated via path 3756A to third party diagnostic/monitoring portion 3702 to be combined (selectively) with sensed parameters (FIG. 19) and/or determined parameters (FIG. 20) to provide feedback 3752 to the stimulation therapy portion 3706 to enhance efficacy of the application of the stimulation therapy.

Similarly, results (e.g. efficacy, parameters, etc.) from the stimulation therapy (via portion 3706) may be communicated via path 3756B to patient management portion 3704 to be combined (selectively) with clinician-directed parameters (from patient management portion 1160 of portal 1155) and/or with patient-directed parameters (from patient remote 1120 and/or patient mobile app 1112) to provide feedback 3754 to the stimulation therapy portion 3706 to enhance efficacy of the application of the stimulation therapy. In some examples, the efficacy may be enhanced via equipping the stimulation therapy portion to make decisions, which may result in automatic adjustment of a stimulation protocol, such as an intensity of stimulation (e.g. amplitude, pulse width, duty cycle, etc.) prior to, or during, application of stimulation signal to target tissue to treat sleep disordered breathing, such as by increasing or maintaining upper airway patency by tongue protrusion, upper airway stiffening, and the like.

In some examples, the feedback 3752 from the third party diagnostic/monitoring portion 3702 and the feedback 3754 from the patient management portion 3704 may be combined (e.g. paths 3703A, 3703B in FIG. 22) or integrated (e.g. via integrator 3710 in FIG. 22) in a complementary manner prior to being an input for closed loop feedback to the stimulation therapy portion 3706.

FIG. 26 is a block diagram schematically representing an example method (and/or example device) for patient management including closed-loop feedback from a stimulation therapy input and a third-party diagnostic/monitoring input. In some examples, the method 3780 (and/or device) may comprise at least some of substantially the same features and attributes as, and/or an example implementation of, the example arrangement 3720 as described in association with at least FIG. 23.

As shown in FIG. 26, the example method 3780 may comprise providing patient management-based care decisions or output (via portion 3704), such as scheduling a followup office visit by the patient, scheduling a sleep study (e.g. home or formal), being communicated via path 3784A to third party diagnostic/monitoring portion 3702 to be combined (selectively) with sensed parameters (FIG. 19) and/or determined parameters (FIG. 20) to provide feedback 3782 to the patient management portion 3704 to further enhance efficacy of the patient management care, etc. such as the above-noted scheduling, etc.

Similarly, results (e.g. efficacy, parameters, etc.) from the patient management (portion 3704) may be communicated via path 3784B to stimulation therapy portion 3706 to be combined (selectively) with therapy efficacy and/or delivery parameters (e.g. from implantable medical device 1125, patient remote 1120 in FIG. 18) to provide feedback 3786 to the patient management portion 3704.

In some examples, the feedback 3782 from the third party diagnostic/monitoring portion 3702 and the feedback 3786 from the stimulation therapy portion 3706 may be combined (e.g. paths 3723A, 3723B in FIG. 23) or integrated (e.g. via integrator 3729 in FIG. 23) in a complementary manner prior to being an input for closed loop feedback to the patient management portion 3704.

FIG. 27A is a block diagram schematically representing an example control portion 4000. In some examples, control portion 4000 provides one example implementation of a control portion forming a part of, implementing, and/or generally managing the example arrangements, including implantable medical devices, patient remote controls, third-party diagnostic/monitoring devices, clinician portals, cloud resources, patient management systems, patient mobile apps, user interfaces, control portion, instructions, workflows, engines, functions, parameters, and/or methods, as described throughout examples of the present disclosure in association with FIGS. 18-28, FIGS. 11-17B, and/or FIGS. 1-10. In some examples, control portion 4000 includes a controller 4002 and a memory 4010. In general terms, controller 4002 of control portion 4000 comprises at least one processor 4004 and associated memories. The controller 4002 is electrically couplable to, and in communication with, memory 4010 to generate control signals to direct operation of at least some of the example arrangements, including implantable medical devices, patient remote controls, third-party diagnostic/monitoring devices, clinician portals, cloud resources, patient management systems, patient mobile apps, user interfaces, control portion, instructions, workflows, engines, functions, parameters, and/or methods, as described throughout examples of the present disclosure. In some examples, these generated control signals include, but are not limited to, employing instructions 4011 and/or information 4012 stored in memory 4010 to at least direct and manage sleep disordered breathing (SDB) patient care in the manner described in at least some examples of the present disclosure (e.g. FIGS. 18-28, FIGS. 1-10, FIGS. 11-17B), such as but not limited, integrating stimulation therapy, patient management, and/or externally monitored patient information. In some instances, the controller 4002 or control portion 4000 may sometimes be referred to as being programmed to perform the above-identified actions, functions, etc.

In response to or based upon commands received via a user interface (e.g. user interface 4040 in FIG. 27B or example user interfaces throughout FIGS. 18-28) and/or via machine readable instructions, controller 4002 generates control signals as described above in accordance with at least some of the examples of the present disclosure. In some examples, controller 4002 is embodied in a general purpose computing device while in some examples, controller 4002 is incorporated into or associated with at least some of the example arrangements, including implantable medical devices, patient remote controls, third-party diagnostic/monitoring devices, clinician portals, cloud resource, patient management systems, patient mobile apps, user interface, control portion, instructions, workflows, engines, functions, parameters, and/or methods, etc. as described throughout examples of the present disclosure.

For purposes of this application, in reference to the controller 4002, the term “processor” shall mean a presently developed or future developed processor (or processing resources) that executes machine readable instructions contained in a memory or that includes circuitry to perform computations. In some examples, execution of the machine readable instructions, such as those provided via memory 4010 of control portion 4000 cause the processor to perform the above-identified actions, such as operating controller 4002 to implement sleep disordered breathing (SDB) care via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 4010. The machine readable instructions may include a sequence of instructions, a processor-executable machine learning model, or the like. In some examples, memory 4010 comprises a computer readable tangible medium providing non-volatile storage of the machine readable instructions executable by a process of controller 4002. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 4002 may be embodied as part of at least one application-specific integrated circuit (ASIC), at least one field-programmable gate array (FPGA), and/or the like. In at least some examples, the controller 4002 is not limited to any specific combination of hardware circuitry and machine readable instructions, nor limited to any particular source for the machine readable instructions executed by the controller 4002.

In some examples, control portion 4000 may be entirely implemented within or by a stand-alone device.

In some examples, the control portion 4000 may be partially implemented in one of the example arrangements, including implantable medical devices, patient remote controls, third-party diagnostic/monitoring devices, clinician portals, cloud resources, patient management systems, patient mobile apps, etc. and partially implemented in a computing resource separate from, and independent of, the example arrangements, including clinician portals, cloud resources, patient management systems, patient mobile apps, etc. but in communication with such example arrangements, etc. For instance, in some examples control portion 4000 may be implemented via a server accessible via the cloud and/or other network pathways. In some examples, the control portion 4000 may be distributed or apportioned among multiple devices or resources such as among a server, components of an example arrangement (e.g. 3100 in FIG. 18 or FIGS. 19-27B), and/or a user interface.

In some examples, control portion 4000 includes, and/or is in communication with, a user interface 4040 as shown in FIG. 27B. In some examples, at least some portions or aspects of the user interface 4040 are provided via a graphical user interface (GUI), and may comprise a display 4042 and input 4044. In some examples, user interface 4040 comprises a user interface or other display that provides for the simultaneous display, activation, and/or operation of at least some of the example arrangements, including implantable medical devices, patient remote controls, third-party diagnostic/monitoring devices, clinician portals, cloud resources, patient management systems, patient mobile apps, user interface, control portion, workflows, instructions, engines, functions, parameters, and/or methods, etc., as described in association with FIGS. 18-28. For instance, the various user interfaces described in association with FIGS. 18-28 may each provide an example implementation of user interface 4040.

FIG. 28 is a flow diagram schematically representing an example method of patient care for treating sleep disordered breathing (SDB). In some examples, method 4050 may be implemented via at least some the example arrangements, including implantable medical devices, patient remote controls, third-party diagnostic/monitoring devices, clinician portals, cloud resources, patient management systems, patient mobile apps, user interfaces, control portion, instructions, workflows, engines, functions, parameters, and/or methods, as described in association with at least FIGS. 18-27B (and/or FIGS. 11-17B, 1-10). In some examples, method 4050 may be implemented via at least some example arrangements, including implantable medical devices, patient remote controls, third-party diagnostic/monitoring devices, clinician portals, cloud resources, patient management systems, patient mobile apps, user interfaces, control portion, instructions, workflows, engines, functions, parameters, and/or methods other than those described in association with at least FIGS. 18-27B (and/or FIGS. 11-17B, 1-10).

As shown at 4052 in FIG. 28, the method 4050 comprise receiving, via a processing resource, information from stimulation therapy activity, from patient management, and from external patient monitoring. As shown at 4054 in FIG. 28, the method 4050 also may comprise integrating, via a processing resource, the information to perform patient care.

In some examples, the method 4050 may further comprise, based on the integrated information, performing patient care via determining a stimulation therapy to be delivered to upper airway patency-related tissue to treat sleep disordered breathing.

In some examples, the method 4050 may further comprise delivering, via a stimulation element, the determined stimulation therapy to the upper airway patency-related tissue.

In some examples, the method 4050 may further comprise the stimulation element comprising an implantable medical device which includes stimulation signal generating circuitry.

In some examples, the method 4050 may further comprise determining the stimulation therapy as adjusting an intensity of the stimulation therapy signal via at least one of an amplitude, a pulse width, and a duty cycle.

In some examples, the method 4050 may further comprise automatically implementing the adjusting of the intensity of the stimulation therapy signal based on at least one of the externally monitored patient information and the patient management information.

In some examples, the method 4050 may further comprise the receiving externally monitored patient information comprising receiving sensed physiologic information including at least one of: cardiac information; respiratory information; chest motion; oxygen desaturation information; peripheral arterial information; blood pressure; body position; and acoustic information including snoring.

In some examples, the method 4050 may further comprise the receiving the externally monitored patient information comprising receiving determined information including at least one of: sleep disorder breathing (SDB)-related index information; sleep information; and cardiac information.

In some examples, the method 450 may further comprise, based on the integrated information, performing patient care via determining patient management actions and communicating the determined patient management actions.

In some examples, the method 4050 may further comprise the patient management actions comprising at least one of: scheduling a patient office visit; implementing at least one of a communication and a command to adjust parameters of the stimulation therapy; implementing at least one of a communication and a command to adjust parameters of the external patient monitoring.

In some examples, the method 4050 may further comprise performing the determined patient management actions via: receiving, via a processing resource, patient information from a patient device; and displaying, via the processing resource, at least some of the patient information on a clinician portal.

In some examples, the method 4050 may further comprise the integrated information comprising the patient information.

In some examples, the method 4050 may further comprise the displaying comprising arranging the clinician portal to be displayable on a user interface of a clinician computing device in communication with the processing resource.

In some examples, the method 4050 may further comprise the receiving from the patient device comprising: receiving the patient information via a communication link between the patient device and the processing resource; and receiving the patient information at the patient device, via a patient app hosted on the patient device.

In some examples, the method 4050 may further comprise receiving, via the patient app, the patient information from a patient remote control, wherein the patient information comprises at least one of: information received from an implantable medical device; and information, from the patient remote control, regarding patient use of the patient remote control.

In some examples, the method 4050 may further comprise receiving, via the patient app, the patient information from a third party device.

In some examples, the method 4050 may further comprise the patient information comprising externally monitored physiologic-related information.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method comprising:
receiving, via a processing resource, information from stimulation therapy activity, from patient management, and from external patient monitoring; and
integrating, via a processing resource, the information to perform patient care.

2. The method of claim 1, comprising:
based on the integrated information, performing patient care via determining a stimulation therapy to be delivered to upper airway patency-related tissue to treat sleep disordered breathing.

3. The method of claim 2, comprising:
delivering, via a stimulation element, the determined stimulation therapy to the upper airway patency-related tissue.

4. The method of claim 3, wherein the stimulation element comprises an implantable medical device which includes stimulation signal generating circuitry.

5. The method of claim 2, wherein determining the stimulation therapy comprising:
adjusting an intensity of the stimulation therapy signal via at least one of an amplitude, a pulse width, and a duty cycle.

6. The method of claim 5, comprising automatically implementing the adjusting of the intensity of the stimulation therapy signal based on at least one of the externally monitored patient information and the patient management information.

7. The method of claim 1, wherein the receiving externally monitored patient information comprises receiving sensed physiologic information including at least one of:
cardiac information;
respiratory information;
chest motion;
oxygen desaturation information;
peripheral arterial information;
blood pressure;
body position; and
acoustic information including snoring.

8. The method of claim 1, wherein the receiving externally monitored patient information comprises receiving determined information including at least one of:
sleep disorder breathing (SDB)-related index information;
sleep information; and
cardiac information.

9. The method of claim 1, comprising:
based on the integrated information, performing patient care via determining patient management actions and communicating the determined patient management actions.

10. The method of claim 9, wherein the patient management actions comprise at least one of:
scheduling a patient office visit;
implementing at least one of a communication and a command to adjust parameters of the stimulation therapy;
implementing at least one of a communication and a command to adjust parameters of the external patient monitoring.

11. The method of claim 9, comprising:
performing the determined patient management actions via:
receiving, via a processing resource, patient information from a patient device; and
displaying, via the processing resource, at least some of the patient information on a clinician portal.

12. The method of claim 11, wherein the integrated information comprises the patient information.

13. The method of claim 11, wherein the displaying comprises:
arranging the clinician portal to be displayable on a user interface of a clinician computing device in communication with the processing resource.

14. The method of claim 11, wherein the receiving from the patient device comprises:
receiving the patient information via a communication link between the patient device and the processing resource; and
receiving the patient information at the patient device, via a patient app hosted on the patient device.

15. The method of claim 14, comprising:
receiving, via the patient app, the patient information from a patient remote control, wherein the patient information comprises at least one of:
information received from an implantable medical device; and
information, from the patient remote control, regarding patient use of the patient remote control.

16. The method of claim 14, comprising:
receiving, via the patient app, the patient information from a third party device.

17. The method of claim 16, wherein the patient information comprises externally monitored physiologic-related information.
